(19) United States
(12) Patent Application Publication
(10) Pub. No.: US 2026/0357352 A1
(43) Pub. Date: Nov. 12, 2026

(54) MACROPHAGE POLARITY AS A SIGNATURE ACROSS CANCERS

(71) Applicants: The General Hospital Corporation, Boston, MA (US); University of Geneva, Geneva (CH)

(72) Inventors: Ralph Weissleder, Peabody, MA (US); Mikael Pittet, Preverenges (CH); Ruben Bill, Wabern (CH); Pratyaksha Wirahpati, Lucens (CH); Sara Pai, Westport, CT (US)

(21) Appl. No.: 19/486,788

(22) PCT Filed: May 21, 2024
(86) PCT No.: PCT/US2024/030379

§ 371 Date: Nov. 21, 2025

Related U.S. Application Data
(60) Provisional application No. 63/503,528, filed on May 22, 2023.

(51) Int. Cl. C12Q 1/6886; C12Q 1/686

(52) U.S. Cl. CPC C12Q 1/6886; C12Q 1/686; C12Q 2600/118; C12Q 2600/158; G01N 2800/52

(57) ABSTRACT

Tumor microenvironments (TMEs) influence cancer progression but are complex and often differ between patients. Considering that microenvironment variations may reveal rules governing intratumoral cellular programs and disease outcome, tumor-to-tumor variation was examiner in 52 head and neck squamous cell carcinomas. macrophage polarity, defined by CXCL9 and SPP1 (CS) expression but not by conventional M1 and M2 markers, had a noticeably strong prognostic association. CS macrophage polarity also identified a highly coordinated network of either pro- or anti-tumor variables, which involved each tumor-associated cell type, and was spatially organized. The methods provided herein use tumor-associated macrophage (TAM) polarity as a variable for cancer prognosis.

### CLAIM OF PRIORITY

This application claims the benefit of U.S. Provisional Application Ser. No. 63/503,528, filed on May 22, 2023. The entire contents of the foregoing are incorporated herein by reference.

### FEDERALLY SPONSORED RESEARCH OR DEVELOPMENT

This invention was made with Government support under Grant No. CA240239, awarded by the National Institutes of Health. The Government has certain rights in the invention.

### TECHNICAL FIELD

The present disclosure relates to using macrophage polarity as a prognostic signature across cancers.

### BACKGROUND

Tumor microenvironments (TMEs) are ecosystems composed of diverse stromal, immune, and cancer cells. The interactions between these components are incompletely understood, but some of them produce pro- or anti-tumor effects (1). It is therefore essential to decipher the components of these ecosystems and better understand some of their internal governance and rules of interaction. The results could lead to an improved understanding of the regulatory mechanisms that determine cancer progression, as well as resistance or response to treatment and, by extension, better define patient prognosis and treatment options. To date, many studies have described TMEs using low-resolution techniques, such as bulk RNA sequencing and immunohistochemistry. These data have allowed classification of TMEs based on broad criteria, such as the composition of the immune infiltrate and the character of the inflammatory response (2). For example, the abundance or phenotype of various immune cells within tumors, including T-cells (3-5), B-cells (6-8), dendritic cells (DCs) (9-11), and macrophages (12-14), may be associated with distinct clinical prognoses. More recently, single-cell RNA sequencing (scRNAseq) is increasingly used to define the TME, as it directly reveals its cellular heterogeneity without the need for predefined sets of markers for cell identification (15-20). Nevertheless, scRNAseq studies typically consider pooled cells from all patients as units of statistical replication, which prevents formal testing of tumor-to-tumor variation between patients.

### SUMMARY

Provided herein are methods of screening for intervention-responsive tumors, which include measuring the expression level of CXCL9 and SPP1 in tumor associated macrophages from a tumor sample; determining a CXCL9:SPP1 ratio; and identifying the tumor sample as originating from an intervention-responsive tumor if the CXCL9:SPP1 ratio is above a reference level.

Additionally, provided herein are methods of determining a ratio between CXCL9 and SPP1 in a tumor sample, which include obtaining a tumor sample; isolating the tumor associated macrophages from the tumor sample; determining expression levels of CXCL9 and SPP1 in the tumor associated macrophages; normalizing expression levels of CXCL9 and SPP1 compared to a housekeeping gene; and comparing the normalized expression level of CXCL9 to the normalized expression level of SPP1 to generate a CXCL9:SPP1 ratio.

Additionally, provided herein are methods of predicting cancer progression in a subject, which include measuring the expression level of CXCL9 and SPP1 in tumor associated macrophages from a tumor sample; determining a CXCL9:SPP1 ratio; identifying the tumor sample as originating from an intervention-responsive tumor if the CXCL9:SPP1 ratio is above a reference level; and using the intervention-responsive tumor status to predict cancer progression in a subject with cancer.

Additionally, provided herein are methods of generating a survival prognosis in a subject with cancer, which include measuring the expression level of CXCL9 and SPP1 in tumor associated macrophages from a tumor sample; determining a CXCL9:SPP1 ratio; and identifying the tumor sample as originating from an intervention-responsive tumor if the CXCL9:SPP1 ratio is above a reference level; and using the intervention-responsive tumor status to generate a survival prognosis in a subject with cancer.

In some embodiments, the methods consist of measuring the expression level of CXCL9 and SPP1 in tumor associated macrophages. In some embodiments, the method further comprises obtaining a tumor sample from the subject. In some embodiments, the sample comprises a solid tumor. In some embodiments, the cancer is a solid tumor cancer. In some embodiments, the solid tumor cancer comprises a sarcoma, a carcinoma, a lymphoma, a germ cell tumor, or a blastoma. In some embodiments, the cancer comprises a chondrosarcoma, a leiomyosarcoma, a rhabdomyosarcoma, a mesothelial sarcoma or mesothelioma, a fibrosarcoma, a angiosarcoma or hemangioendothelioma, a liposarcoma, a glioma, an astrocytoma, a myxosarcoma, a mesenchymouse or mixed mesodermal cancer. In some embodiments, the cancer comprises an adenocarcinoma or a squamous cell carcinoma. In some embodiments, the cancer is a skin cancer, a brain cancer, a liver cancer, a pancreatic cancer, a kidney cancer, a bladder cancer, a thyroid cancer, a stomach cancer, a lung cancer, a head or neck cancer, an eye cancer, a lip or oral cancer, a gastrointestinal cancer, a colorectal cancer, an anal cancer, a breast cancer, a cervical cancer, an endometrial cancer, an ovarian cancer, an esophageal cancer, a thymoma, a bone cancer, a lymph node cancer, a prostate cancer, a testicular cancer, a penile cancer, a gallbladder cancer, a uterine sarcoma, an adrenal gland cancer, an ampullary cancer, a nasal or a paranasal sinus cancer. In some embodiments, the cancer is a head or neck cancer. In some embodiments, the CXCL9:SPP1 ratio reference level is at or greater than 1. In some embodiments, the expression levels of CXCL9 and SPP1 are normalized to a housekeeping gene prior to determining the CXCL9:SPP1 ratio. Some embodiments include comparing the normalized level of CXCL9 expression to the normalized level of SPP1 expression in the sample. In some embodiments, the expression level of CXCL9 and SPP1 is determined by immunohistochemical methods. In some embodiments, the expression level of CXCL9 and SPP1 is determined by qPCR. Some embodiments further comprise treating the subject having an intervention-responsive tumor by administering to the subject an immunotherapy agent. In some embodiments, the immunotherapy agent is a checkpoint blockade immunotherapy agent. In some embodiments, the checkpoint blockade immunotherapy agent is an anti-PD1 immunotherapy agent.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1N. CXCL9:SPP1 polarity of tumor-associated macrophages is a major contributor to the clinical outcome of head and neck squamous cell carcinomas (HNSCCs).

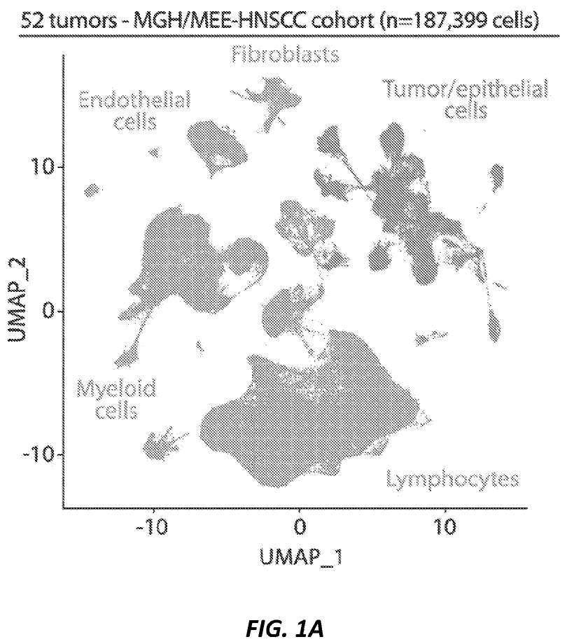

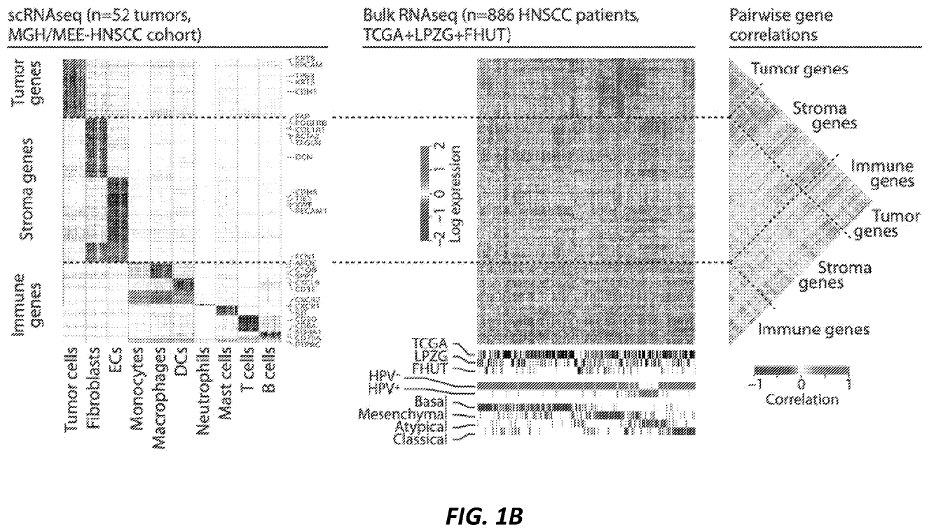

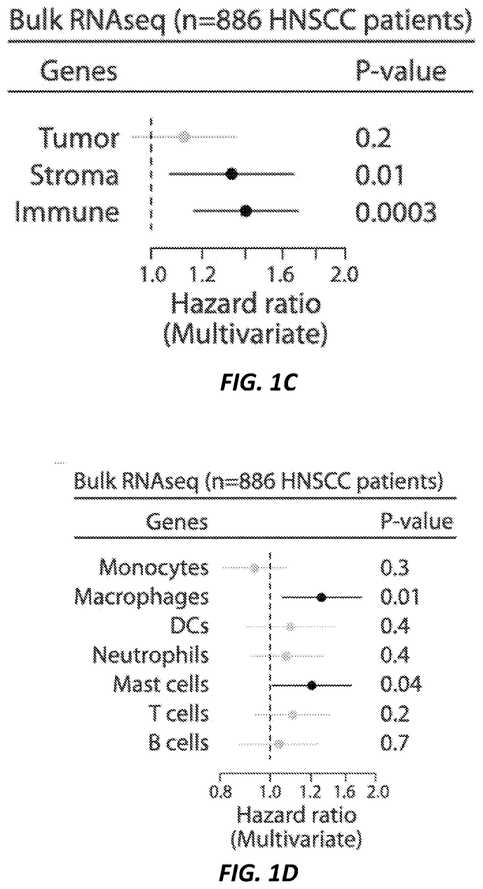

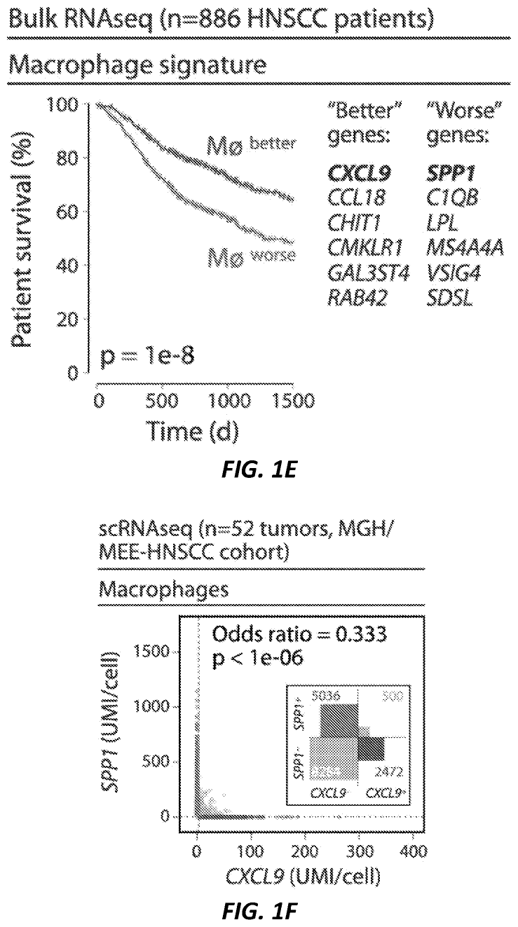

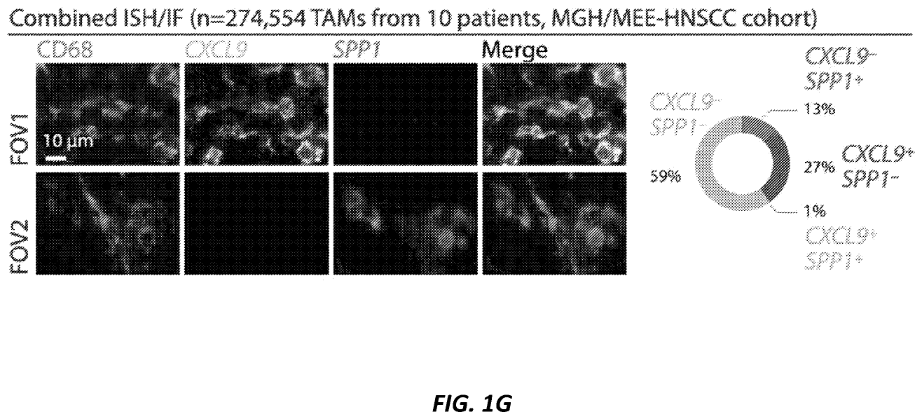

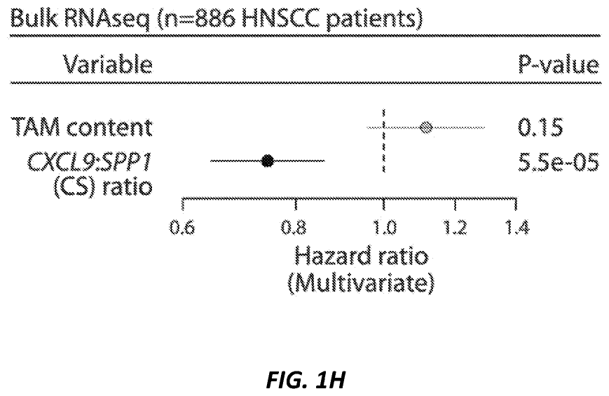

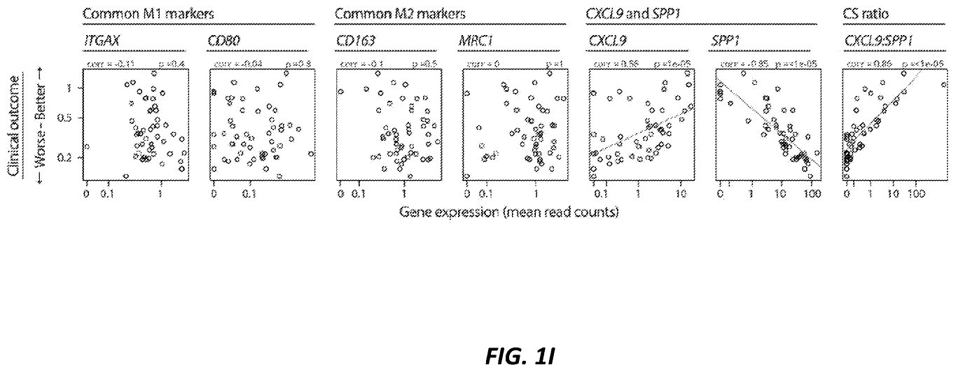

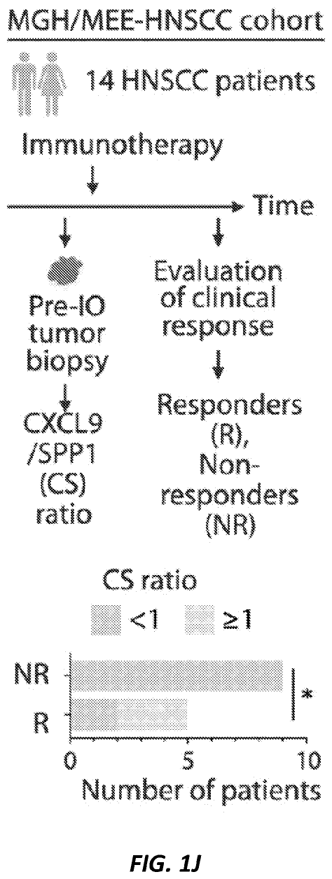

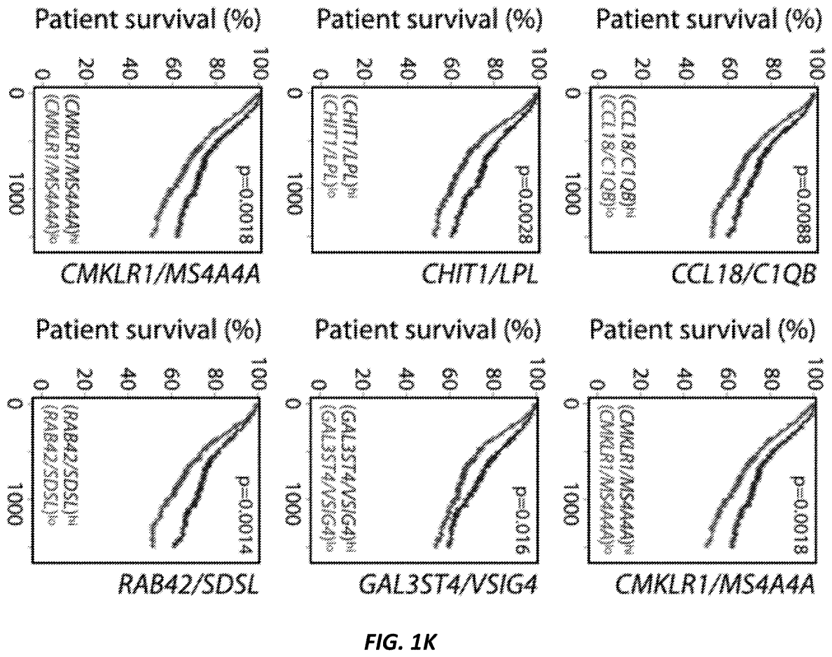

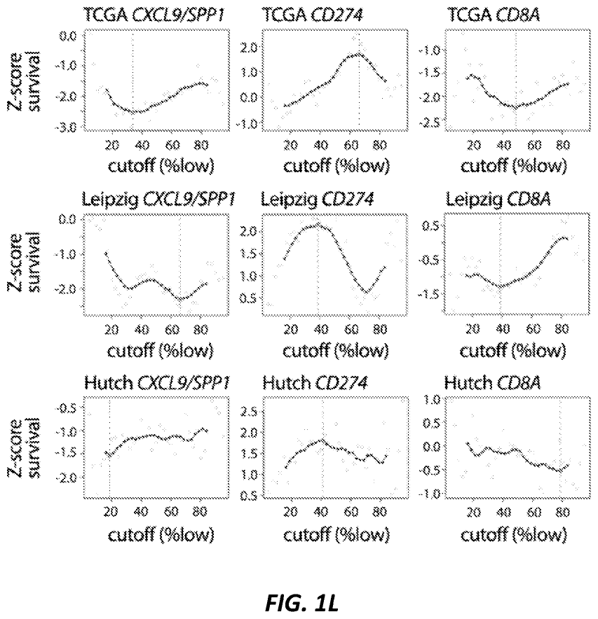

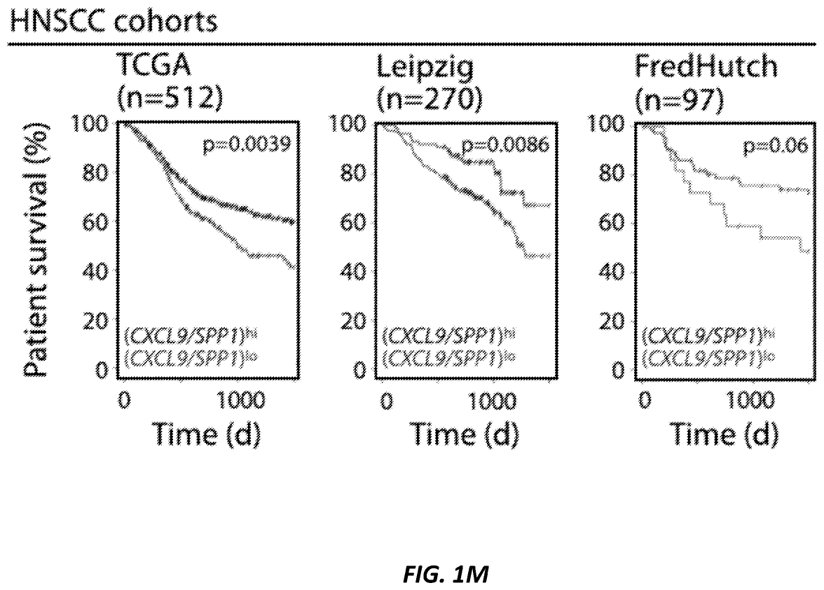

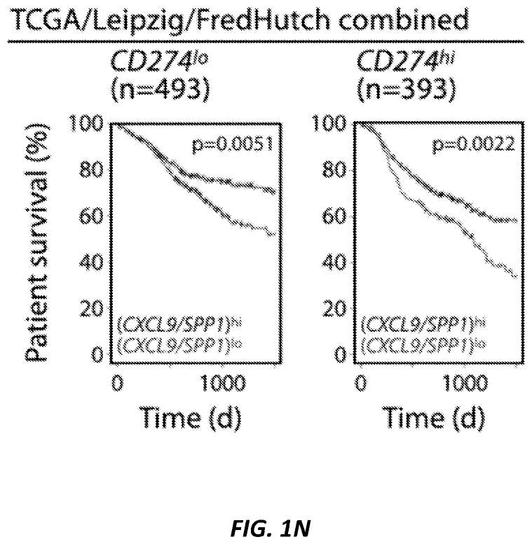

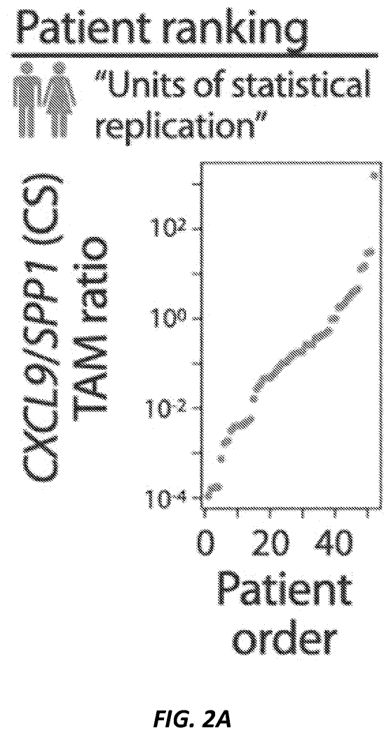

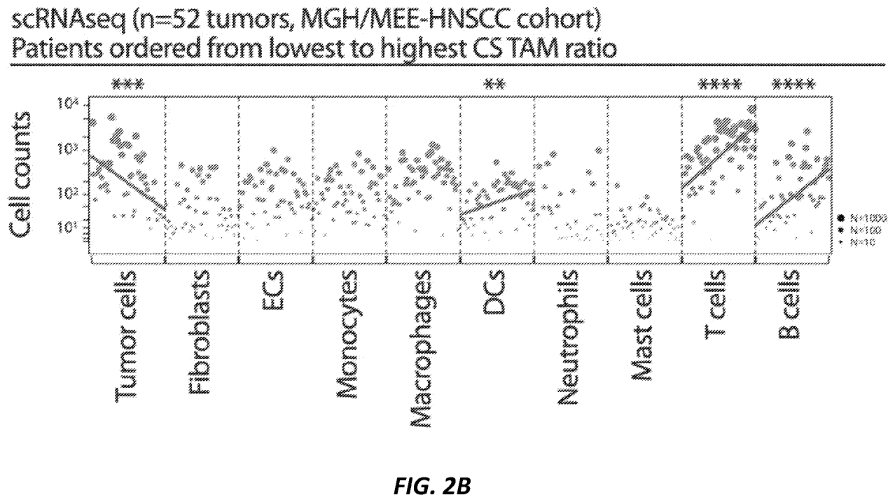

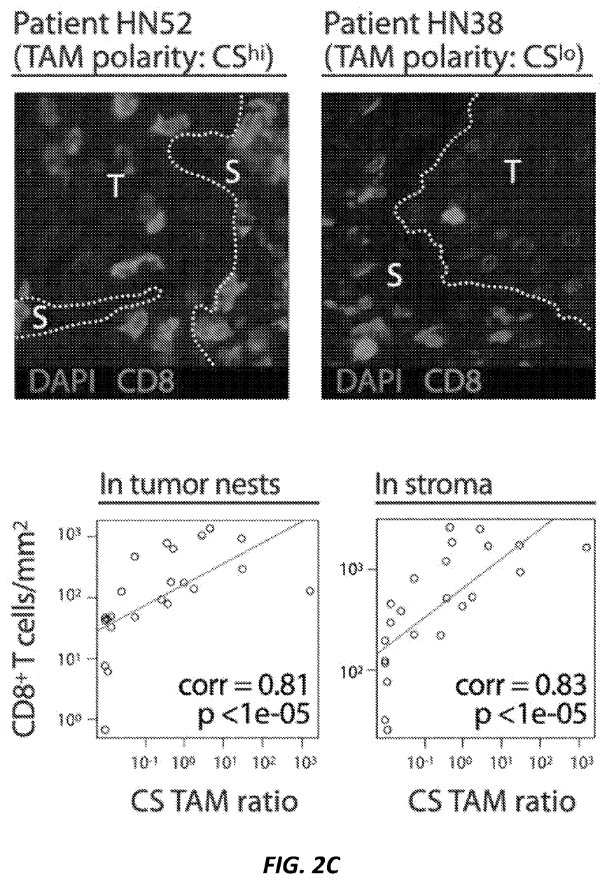

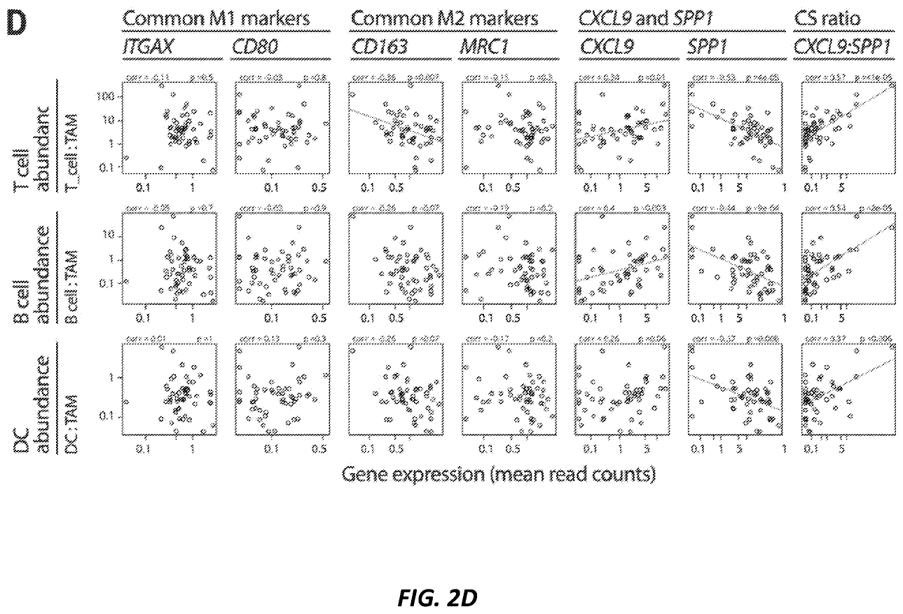

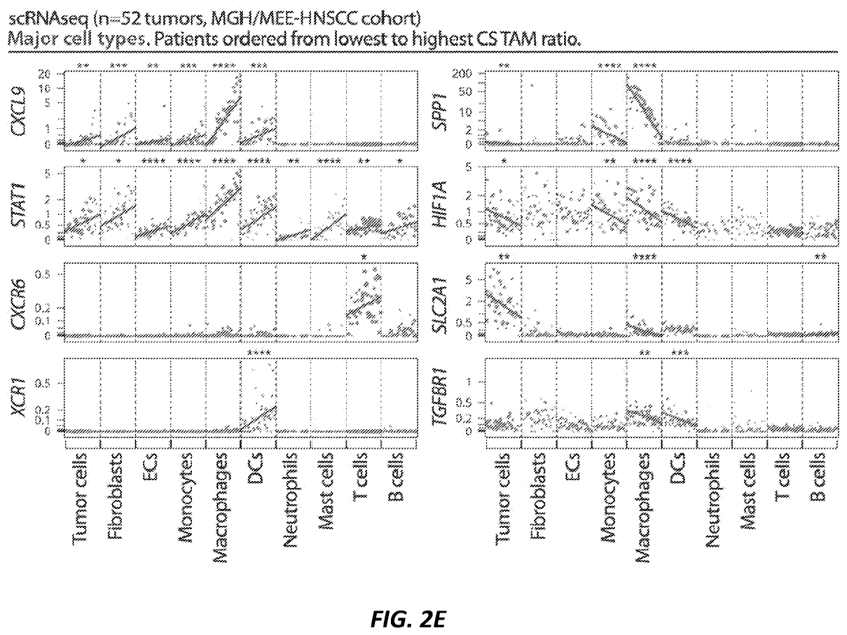

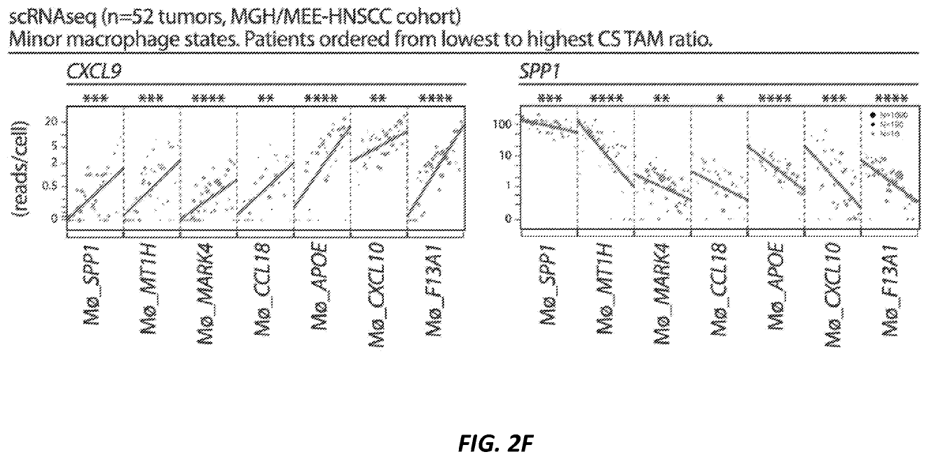

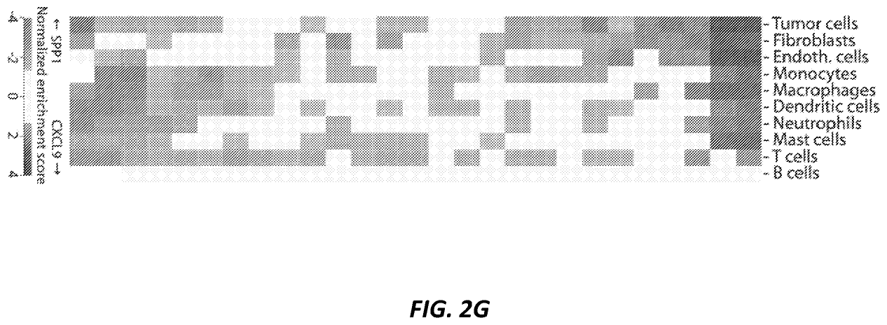

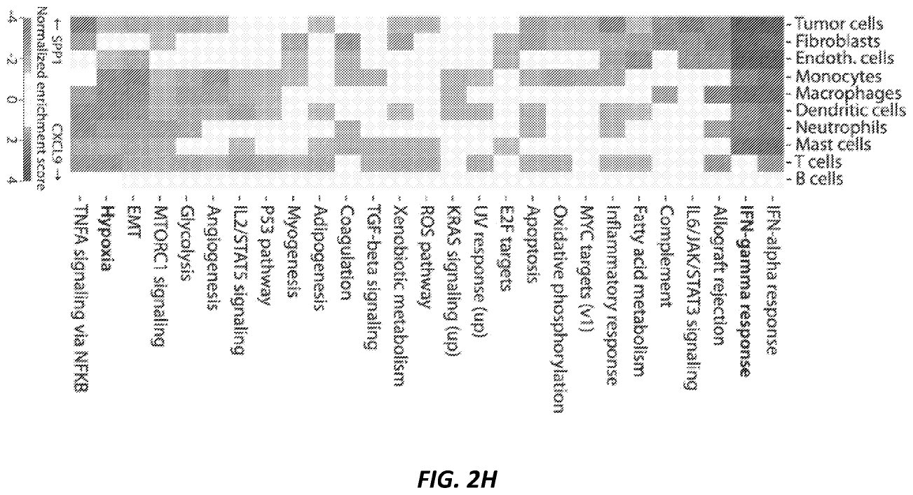

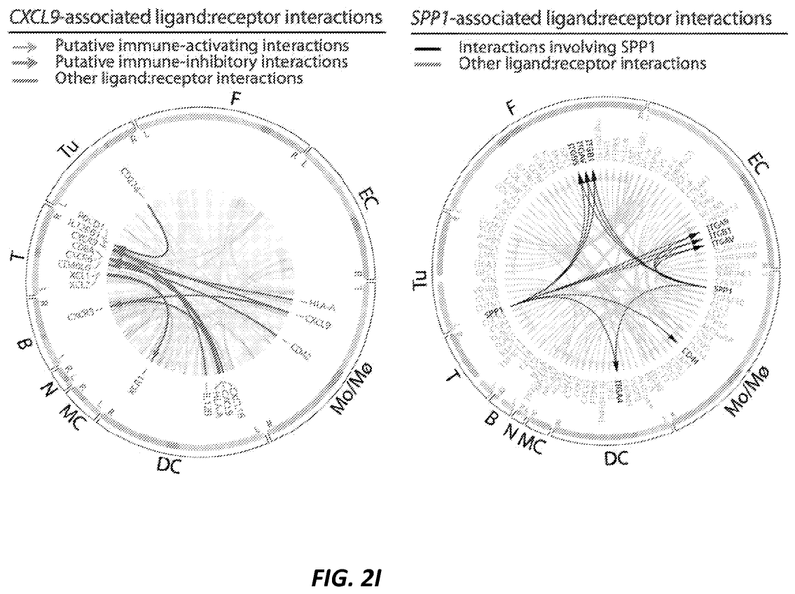

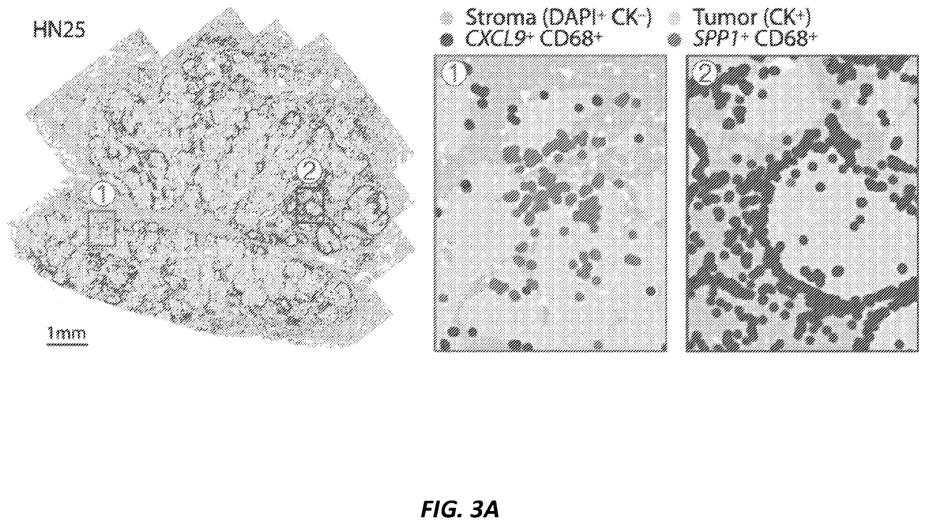

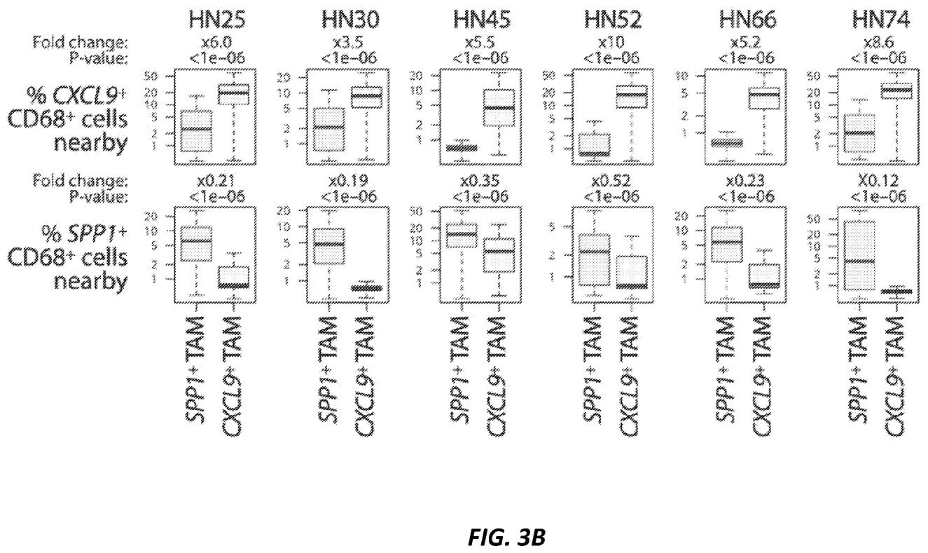

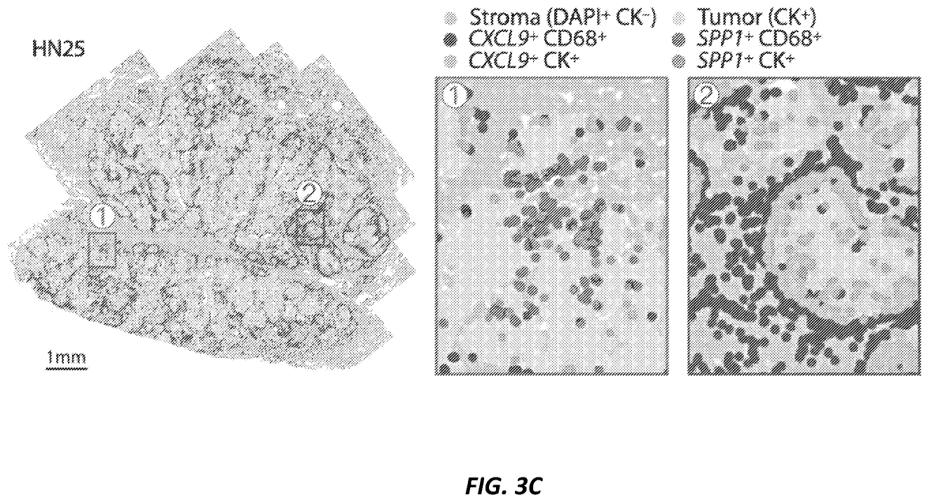

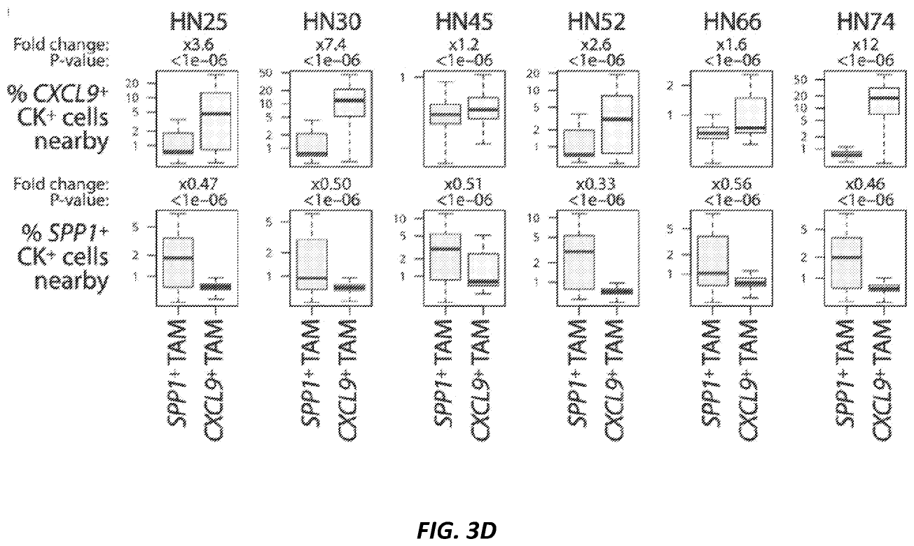

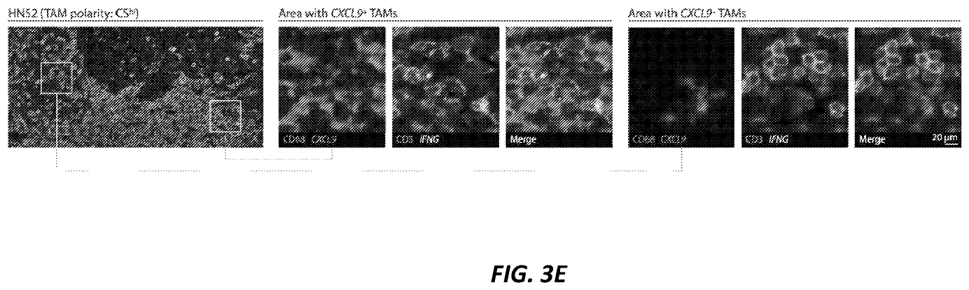

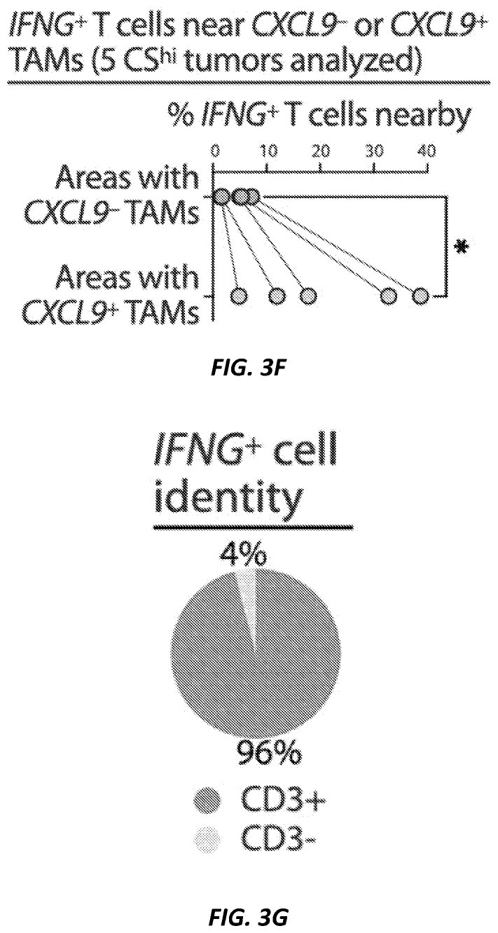

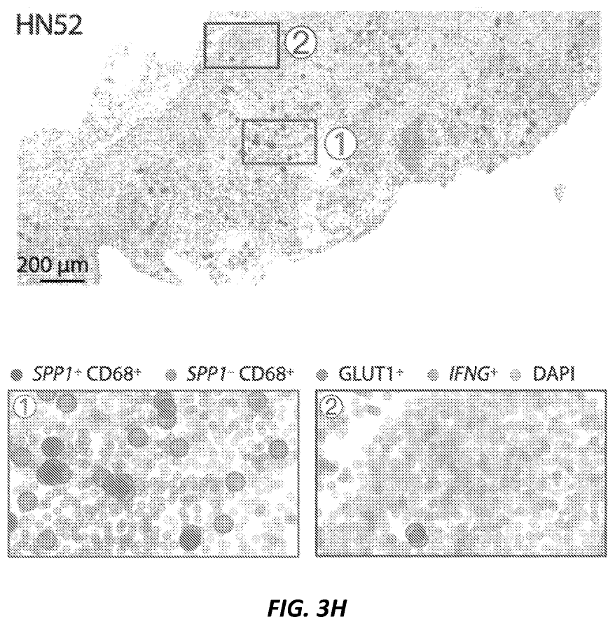

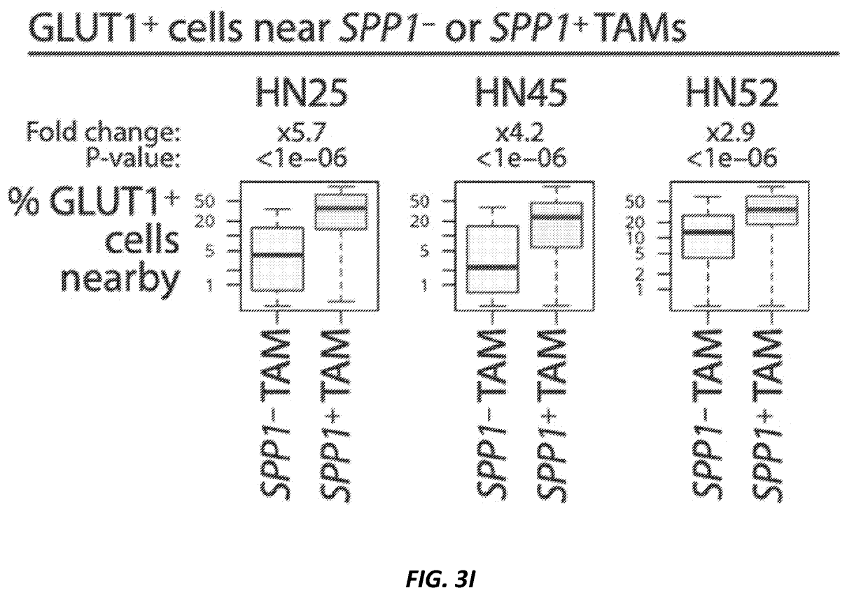

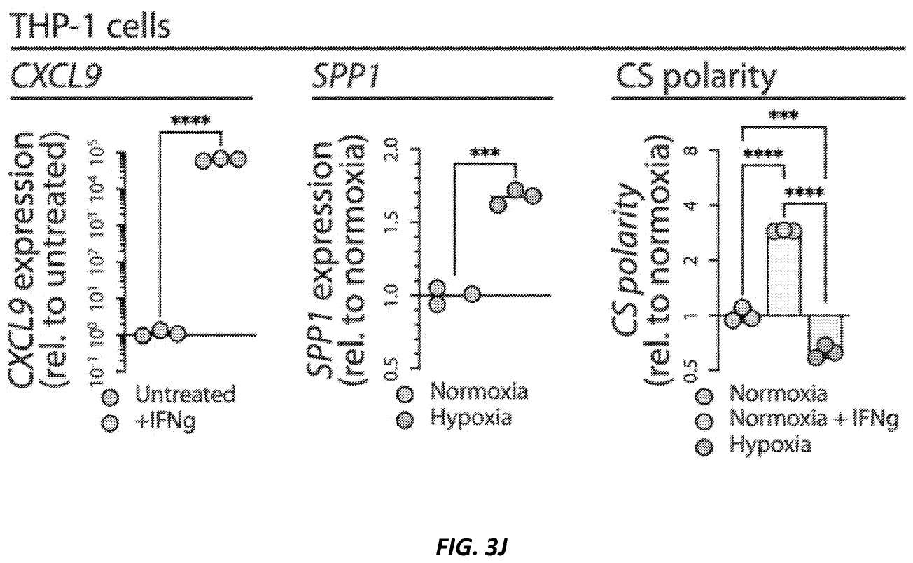

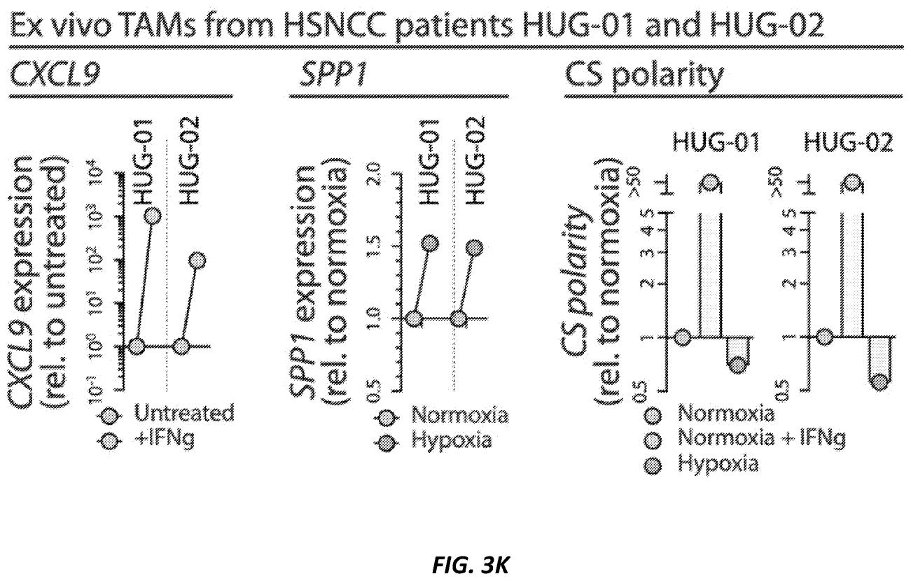

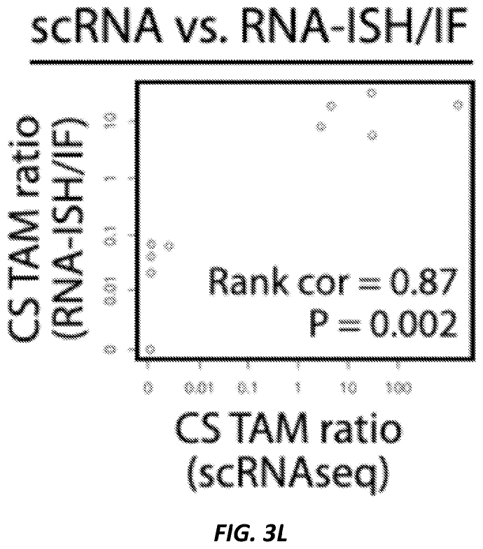

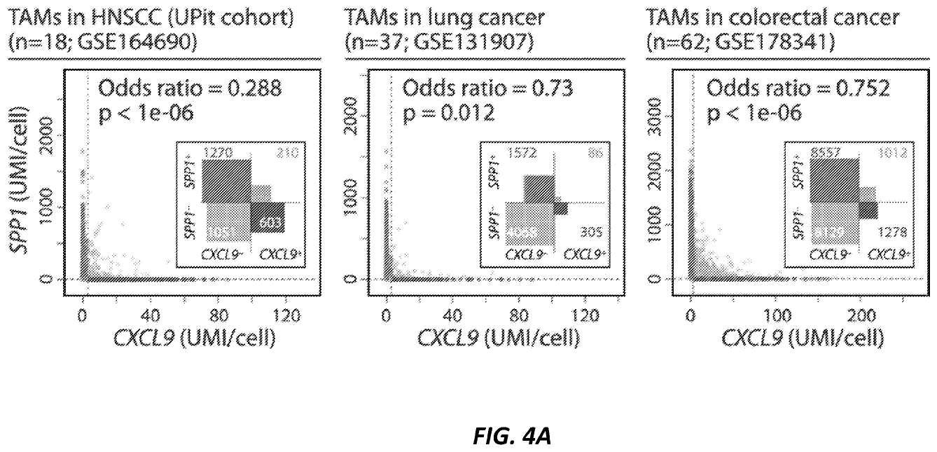

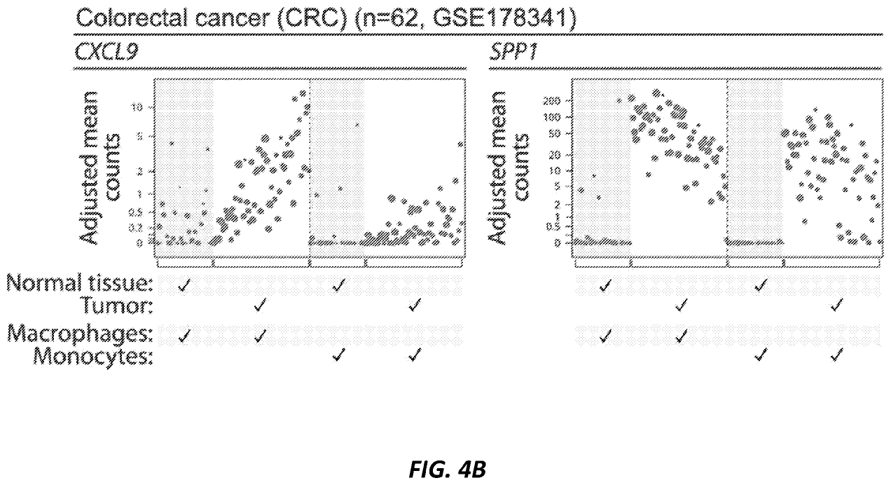

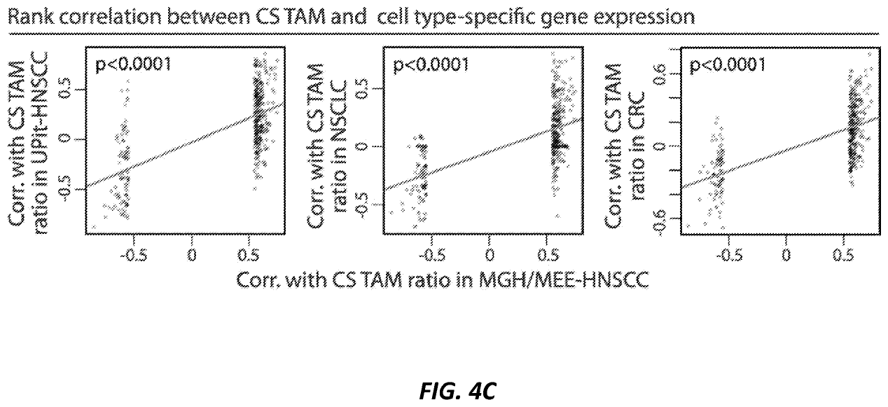

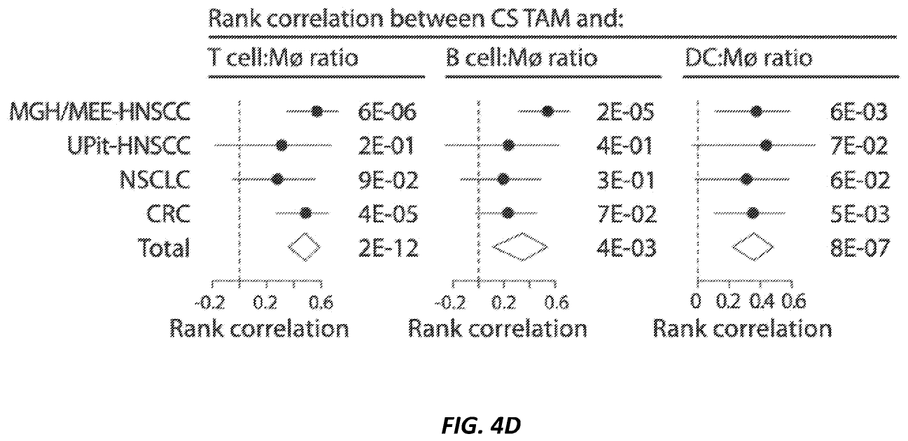

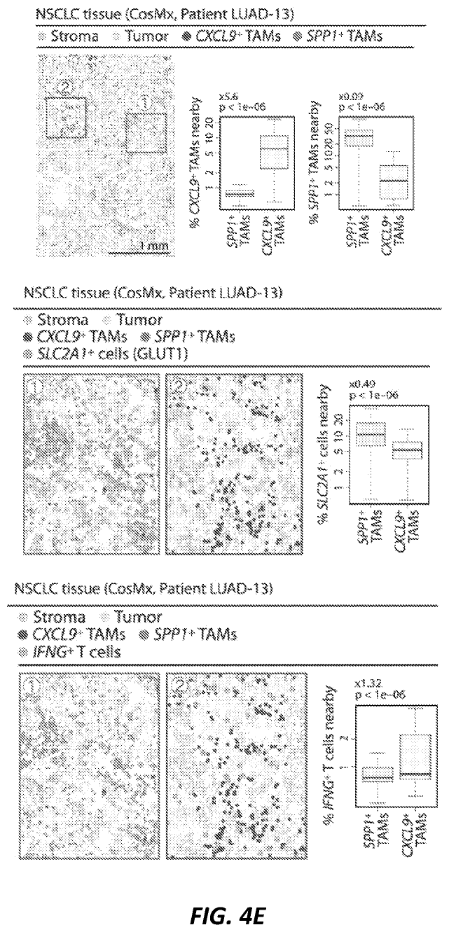

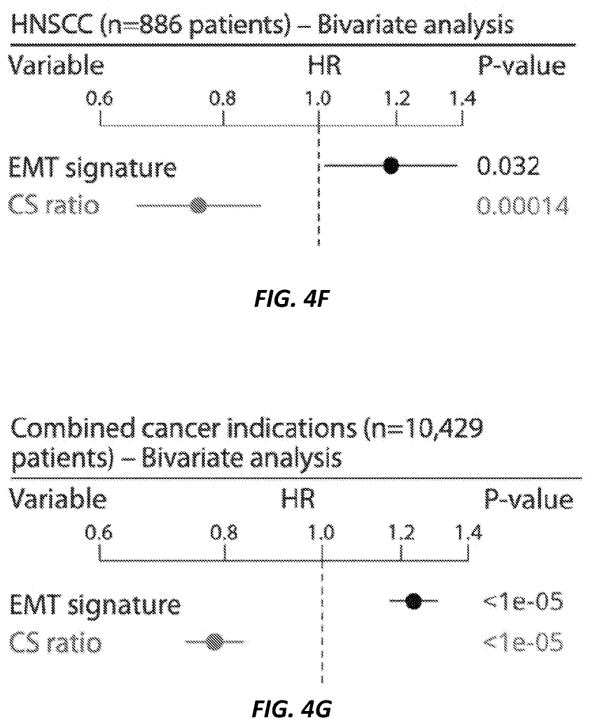

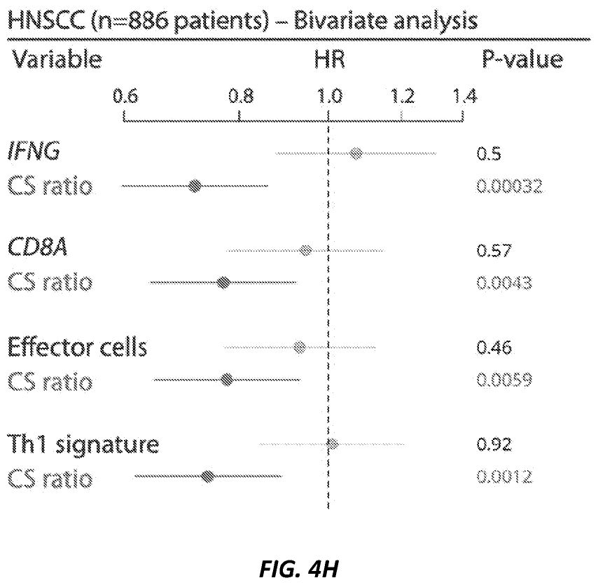

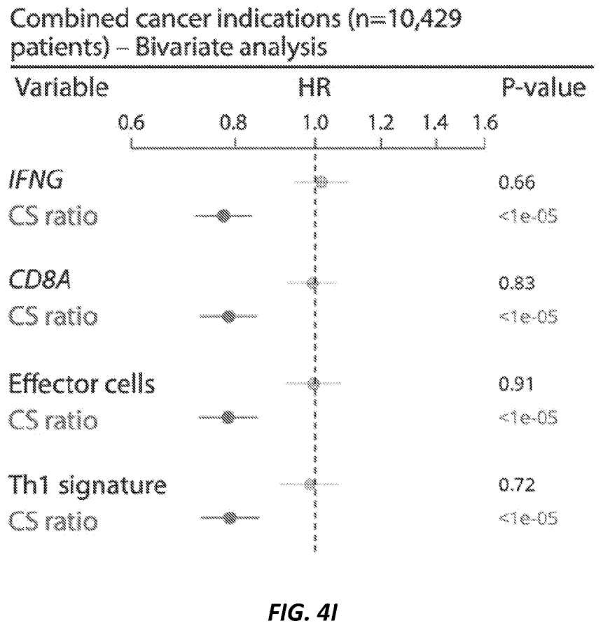

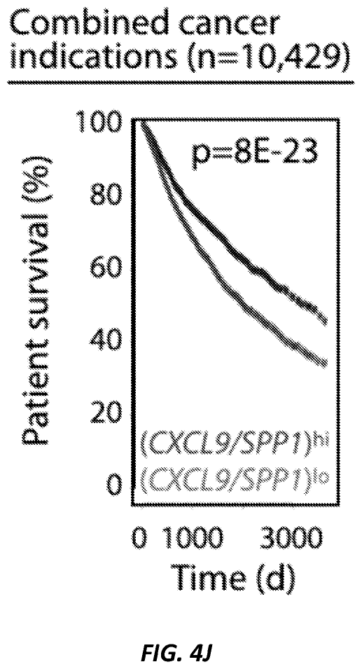

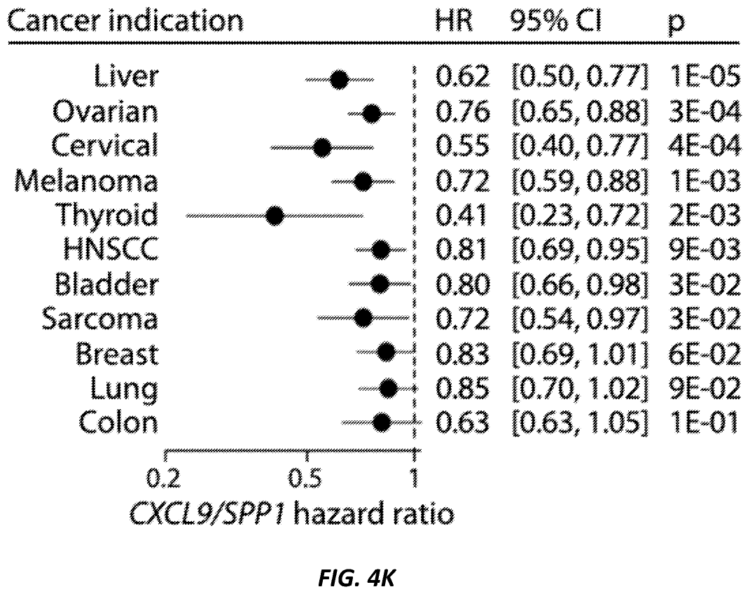

(FIG. 1A) Uniform manifold approximation and projection (UMAP) visualization of single-cell transcriptomic profiles combined from all 52 samples from the MGH/MEE-HNSCC cohort, highlighting separation of main cell lineages and showing finer clustering within each. (FIG. 1B) Identification of genes that are dominantly expressed (with fold-change>3 relative to the next most-expressing cell type; multiple-test testing p-value<0.05 for patient-level comparison) in each major cell type, and based on scRNAseq data from the MGH/MEE-HNSCC cohort (left panel). Projection of these genes to bulk RNAseq data from public datasets (totalling n=886 HNSCC patients) shows their relative expression pattern together with associated cohort of origin, HPV status and molecular subtypes (middle panel). Pairwise correlations between the same genes (right panel). The patients were sorted according to hierarchical clustering based on the tumor-specific genes only. (FIG. 1C) Comparison of three prognostic signatures, each derived using tumor, stromal and immune genes only. Multivariable Cox regression was used to obtain the hazard ratios (with Wald 95% confidence intervals, shown as horizontal bars, and p-values), based on the cross-validated prognostic scores derived using the GLMNET Cox model and applied to pairwise differences of expression of the genes. (FIG. 1D) Similar analysis as in (C), but using genes dominant in each subdivision of the immune cell types. (FIG. 1E) Kaplan-Meier plot of cross-validated macrophage prognostic score from (D) at 50% cutoff, showing the genes with opposite effects selected by the model. (FIG. 1F) Scatter plot of CXCL9 and SPP1 expression in each of 16,292 macrophages, and a contingency table based on dichotomized expression (positive if UMI counts>3), with odds ratio and Fisher's exact test p-value to indicate mutual exclusion (odds ratio<1). (FIG. 1G) Histological analysis with combined RNA-ISH/IF, using RNA probes for CXCL9 and SPP1 and antibody marker for macrophages (CD68) on 10 samples from the MGH/MEE-HNSCC cohort. Representative histology images (left) and quantification of TAM positive or not for CXCL9 and/or SPP1 (right) are shown. (FIG. 1H) Cox regression analysis in bulk RNAseq cohorts (as in FIGS. 1B and 1E) comparing the prognostic impact of TAM abundance signature (derived from the pseudo-bulk profiles of the scRNAseq data using GLMNET classifiers) and the CXCL9:SPP1 (CS) ratio. Wald 95% confidence intervals and p-values were shown. (FIG. 1I) Scatter plots showing lack of correlations between the macrophage signature score (FIG. 1D-E) and common M1/M2 markers, and substantial correlations between the whole signature with individual expression of CXCL9 and SPP1, as well as the CS ratio. Spearman's rank correlation is used and when significant a fitted red line is shown. (FIG. 1J) Association between CS ratio and response to an anti-PD1 mAb-containing treatment regimen in 14 patients of the MGH/MEE-HNSCC cohort, with significant Fisher's exact test. (FIG. 1K) Kaplan-Meier survival analysis of pooled HNSCC bulk RNAseq datasets of the prognostic gene pairs in macrophages as in FIG. 1E, with 50% cutoff for dichotomization. (FIG. 1L) Dataset-specific optimal selection of cutoffs for CXCL9/SPP1 ratio, CD274 and CD8A expression, based on Cox regression Z-scores at a range of possible splits in percentage of low-and high-risk groups. (FIG. 1M) Kaplan-Meier estimates of overall survival in three publicly available HNSCC bulk RNAseq datasets (TCGA, LPZG, FHUT). Patients were stratified according to bulk CXCL9/SPP1 ratio with optimized thresholds as shown in FIG. 1L. (FIG. 1N) Adjustment of CXCL9/SPP1 ratio by CD274 bulk mRNA expression, shown as stratified Kaplan-Meier plots.

FIGS. 2A-2A. CXCL9:SPP1 tumor-associated macrophage polarity determines the broader tme.

(FIG. 2A) Patient ranking according to the CS TAM ratio. (FIG. 2B) Cell counts of major cell types. Patients were ranked from lowest to highest CS TAM ratio. Significant correlation based on Spearman's rank correlation with CS TAM is indicated with a red line. Each dot represents the value for one sample with the dot size being representative of the cell number contributing to that value. (FIG. 2C) Quantitative immunofluorescence on 23 samples showing correlation between CS TAM ratio and CD8⁺ T-cell abundance in both tumor nest and stroma. (FIG. 2D) Correlations between T, B and dendritic cell abundance (relative to TAM counts) and common M1/M2 markers, CXCL9, SPP1 and CS ratio. Spearman rank correlation is used, and when significant, a fitted red line is shown. (FIG. 2E) Cell-type- and patient-specific expression of example genes, plotted according to patient order defined by CS ratio. The left panels show genes that are positively correlated with CS ratio, and the right panels show negatively correlated genes. (FIG. 2F) Association between CS TAM polarity and the expression of CXCL9 (left), and SPP1 (right) in minor TAM states. (FIG. 2G) Heat-map summarizing the correlation analysis (similar to FIG. 2E) when applied to 195 cytokines in all cell types and indicating those that are significant in at least one cell type (FDR<0.05). (FIG. 2H) Heat-map of gene set enrichment analysis results, applied separately to each cell type and taking as the input the correlation with CS ratio of FIG. 2F, showing significant MSigDB hallmark gene sets. (FIG. 2I) Circos plots showing well-known ligand-receptor pairs from curated databases (as compiled in the NicheNet software package) under the requirement that either the ligand or the receptor (or both) is correlated with CS ratio, in addition to both being expressed. Arrows are pointing from the ligand towards the receptors. Only selected example pairs are labeled and highlighted for CXCL9 network. Abbreviations: Tu: tumor, T: T-cells, B: B-cells, N: neutrophils, MC: mast cells, DC: dendritic cells, Mo/Mø: monocyte/macrophages, EC endothelial cells, F fibroblasts. ****p<0.0001, ***p<0.001, **p<0.01, *<0.05. P-values are adjusted for the number of cell types. ECs: endothelial cells, DCs: dendritic cells.

FIGS. 3A-3L. IFNγ and hypoxia respectively increase and decrease CXCL9:SPP1tumor-associated macrophage polarity.

(FIG. 3A) “Pseudo-image” of summarized biomarker status per cell from a combined RNA-ISH and IF histology from a HNSCC tissue sample. Each one of the 650,340 cells is plotted as a dot located at the center of the original cell image, colored according to categorized biomarker combinations. Two magnified regions highlighting concentration of CXCL9⁺ or SPP1⁺ TAMs are shown to the right. (FIG. 3B) Quantitative analysis comparing the density of the CXCL9⁺ TAMs (top row) and SPP1⁺ TAMs (bottom) in the neighborhood surrounding every SPP1⁺ or CXCL9⁺ TAM cell (horizontal axis). The neighborhood is defined as four layers of cells according to Delaunay triangulation (with approximately ~40 μm distance to the outmost layer and a total of ~70 cells per neighborhood). The percentage of the cell type of interest is compared by t-test (summarized by the fold change and p-value). The box plots show Tukey's lower and upper hinges, the quartiles and the median. Data from 6 samples were analyzed in the same manner. (FIG. 3C) Similar plot to FIG. 3A, adding CK⁺ tumor cells that expresses CXCL9 or SPP1. (FIG. 3D) Similar analysis to FIG. 3B but comparing the percentage of CXCL9⁺ or SPP1⁺ tumor cells in the neighborhood surrounding CXCL9⁺ or SPP1⁺ TAMs. (FIG. 3E) Histology image from a HNSCC sample, with antibody markers for TAMs (CD68) and T-cells (CD3), as well as RNA-ISH probes for CXCL9 and IFNG. Areas with different CXCL9⁺ TAMs densities are magnified, showing enrichment of IFNG⁺ CD3⁺ cells around CXCL9⁺ TAMs. (FIG. 3F) Quantitative analysis of the histological images as in FIG. 3E from 5 patients with CS^{hi} score, showing consistent enrichment of IFNG⁺ T-cells in areas with high CXCL9⁺ TAM density. (FIG. 3G) Percentage of CD3⁺ cells among IFNG⁺ cells. (FIG. 3H) RNA-ISH-IF analysis of IFNG and hypoxia marker GLUT1 in the context of SPP1⁺ and SPP1⁻ TAMs. (FIG. 3I) Quantitative analysis comparing GLUT1 occurrence in the neighborhood of SPP1⁺ versus SPP1⁻ TAMs, in samples from 3 patients (with the same method as that for FIGS. 3B and 3D). (FIG. 3J) Impact of IFNγ and hypoxia on the expression of CXCL9 and SPP1 by THP-1macrophage-like cells. Cells were exposed to 40 ng/ml of IFNγ or 1 % O₂ for 24 hours. Expression of CXCL9 and SPP1 was measured by RT-qPCR and the combined CS ratio was calculated. Shown are technical triplicates representative of one out of two independent experiments. (FIG. 3K) Impact of IFNγ or hypoxia on the expression of CXCL9 and SPP1 by TAMs obtained from two primary human HNSCC samples (HUG-01 and HUG-02). The cells were obtained by enzymatic digestion of fresh biopsies immediately exposed to 40 ng/ml of IFNγ or 1 % O₂. TAMs were FACS-enriched after 72 hours for analysis of CXCL9 and SPP1 content by RT-qPCR. (FIG. 3L) CS ratio in CD68+ TAMs, determined by combined RNA-ISH and immunofluorescence (IF), was compared to the CS TAM ratio determined by scRNAseq on the same tumor samples. Ten tumor samples were used, included five that were defined as CShi by scRNAseq, and five others that were defined as 5 CS1o (also by scRNAseq). Spearman's rank correlation analysis was used.

FIGS. 4A-4K. CXCL9:SPP1 tumor-associated macrophage polarity identifies a coordinated network of cellular programs in a wide range of human cancers. (FIG. 4A) Scatter plot of CXCL9 and SPP1 expression in single TAMs from three independent scRNAseq datasets, including HNSCC (UPit cohort), lung and colorectal cancer patients. The number of patients for each cohort is indicated above. The inserts show contingency tables based on dichotomized expression (positive if UMI counts >3), with odds ratio and Fisher's exact test p-value to indicate mutual exclusion (odds ratio <1). (FIG. 4B) CXCL9 and SPP1 expression in macrophages or monocytes, from tumor or adjacent normal tissue, in colorectal cancer. Healthy tissue is highlighted in green. (FIG. 4C) Comparison between cell-type specific gene expression correlations with CS ratio in the MGH/MEE-HNSCC cohort and the corresponding correlations in three independent scRNAseq datasets, quantified by the “correlation of correlations” (p-value by Spearman's rank correlation test). (FIG. 4D) Rank correlation between CS TAM polarity and relative abundance of T, B and dendritic cells (FIG. 4D), on the three independent scRNAseq datasets. P-values are indicated to the right of the forest plot and the 95% confidence intervals are shown as horizontal bars. The totals are summarized by random-effect meta-analysis. (FIG. 4E) Spatial transcriptomics analysis of CosMx lung cancer data, showing spatial separation of CXCL9⁺ versus SPP1⁺ TAMs density in the same tumor sample (left panel), quantified by the same analysis as in FIG. 3B. Enrichment of SLC2A1⁺ cells around SPP1⁺ TAMs and INFG⁺ T-cells around CXCL9⁺ TAMs are also observed (middle and right panel, respectively). (FIG. 4F) Assessment of overall survival prognostic impact of bulk RNAseq CS ratio in HNSCC cohorts (as in FIGS. 1B-1E), simultaneously with an EMT signature as competing explanatory variable in Cox regression analysis (HR: hazard ratios, Wald 95% confidence intervals are shown as horizontal bars). (FIG. 4G) As in (FIG. 4F), applied to TCGA pan-cancer collection. (FIG. 4H) As in (FIG. 4F), on HNSCC cohorts comparing CS ratio pairwise to single-gene expression of IFNG and CD8A, as well as the effector cells and Th1 signatures from Bagaev's TME signature collection. (FIG. 4I) As in (FIG. 4H) applied to TCGA pan-cancer collection. (FIG. 4J) Kaplan-Meier estimates of overall survival between CS^{hi} TAM and CS^{lo} TAM patients across 33 different cancer entities in the TCGA pan-cancer collection (n=10,429 patients). (FIG. 4K) Forest-plot corresponding to selected cohorts from (A) with substantial prognostic impacts, indicated by the Hazard ratios (HR), 95% confidence interval (95% CI) and P-values based on Cox regression model.

### DETAILED DESCRIPTION

Through a focused, clinically-driven approach to single-cell analysis of head and neck squamous cell carcinomas (HNSCCs), the inventors have addressed a major difficulty in human disease research, namely the very different manifestation of the same condition from patient to patient, and gained biological insight on tumor associated macrophage (TAM)-related transcriptional co-regulation across cell types in the tumor microenvironment (TME). Specifically, the inventors show herein that cancer patient disease outcome is closely linked to CXCL9 and SPP1 (CS) TAM polarization, and that the latter, although independent of cell differentiation, exhibits plasticity and is strongly linked to the polarization of other immune, stromal, and tumor cells within the TME. CS TAM polarity identifies TME-wide coordination of pro-and anti-tumor pathways. The broad impact of these findings is highlighted in the identified relevance of this variable across multiple solid cancers.

The methods of evaluating the CS axis as described herein are not restricted to any particular TAM state. Therefore, instead of differentiating into highly polarized states, all sublineages of TAMs can activate the CS axis more or less strongly. This is consistent with the memoryless phenotypic plasticity that macrophages exhibit in vitro in response to different stimuli (29) and suggests that pathways associated with CS regulation can be transiently activated in many cell types in response to orchestrated extracellular signaling networks independent of intrinsic lineage differentiation programs. Provided herein are methods of using the ratio of two genes, which may be measured by histology or qPCR analysis. The methods described herein reveal that CS TAM polarity identifies a large co-regulated network of cellular states that control human cancers, and is thus a simple, but critical, determinant of the TME.

### Subjects

The methods described herein use a CXCL9:SPP1 ratio biomarker to determine the prognosis of a particular subject (e.g., the biomarker can be used to screen for intervention-responsive cancer in subjects with a cancer). In some embodiments, the subject has a solid tumor cancer (e.g., the cancer may have metastasized or the cancer may not be metastatic). In some embodiments, subjects with an intervention-responsive cancer are administered a cancer therapy, wherein the cancer therapy can include immunotherapy, chemotherapy, or combinations thereof. In some embodiments, the subject is a mammal. For example, the mammals can be human or non-human veterinary subjects.

### Cancer

Provided herein are methods for identifying an intervention-responsive cancer in a subject with cancer. Cancer and/or tumor refers to a disease, disorder, or condition in which cells exhibit or exhibited relatively abnormal, uncontrolled, and/or autonomous growth, so that they display or displayed an abnormally elevated proliferation rate and/or aberrant growth phenotype. In some embodiments, a cancer can include one or more tumors. In some embodiments, a cancer can be or include cells that are malignant, pre-metastatic, metastatic, and/or non-metastatic. In some embodiments, a cancer can be or include a solid tumor.

In some embodiments, the cancer can be identified as arising from a particular tissue type. For example, the cancer can be identified as a sarcoma, a carcinoma, a lymphoma, a germ cell tumor, or a blastoma. In other embodiments, the cancer can be identified as arising from a tissue of origin or organ of origin. For example, the cancer can be a skin cancer, a brain cancer, a liver cancer, a pancreatic cancer, a kidney cancer, a bladder cancer, a thyroid cancer, a stomach cancer, a lung cancer, a head or neck cancer, an eye cancer, a lip or oral cancer, a gastrointestinal cancer, a colorectal cancer, an anal cancer, a breast cancer, a cervical cancer, an endometrial cancer, an ovarian cancer, an esophageal cancer, a thymoma, a bone cancer, a lymph node cancer, a prostate cancer, a testicular cancer, a penile cancer, a gallbladder cancer, a uterine sarcoma, an adrenal gland cancer, an ampullary cancer, a nasal or a paranasal sinus cancer.

Provided herein are methods of identifying a cancer (e.g., solid tumor cancer) in a subject with cancer that is unlikely to respond to immunotherapy or chemotherapy treatments, wherein the method includes withholding immunotherapy or chemotherapy for those subjects identified as having a cancer that is not likely to respond to immunotherapy or chemotherapy. Surgery or radiotherapy may be recommended to subjects identified as having a cancer that is not likely to respond to immunotherapy or chemotherapy.

### CXCL9:SPP1 Ratio in Tumor Associated Macrophages as a Biomarker

CXCL9 is a small cytokine within the CXC chemokine family which plays a role in chemotaxis, differentiation, and tissue extravasation, which is encoded by the CXCL9 gene. The SPP1 gene encodes osteopontin (OPN), also known as bone/sialoprotein (BSP-1 or BNSP) which is a heavily phosphorylated extracellular matrix protein. The ratio of CXCL9 and SPP1 expression in tumor associated macrophages from a cancer sample is a prognostic biomarker for the methods presented herein, wherein a biomarker can be predictive, prognostic, and/or diagnostic, of a relevant biological event or state of interest. Those of skill in the art will appreciate that a biomarker may be individually determinative of a particular biological event or state of interest or may represent or contribute to a determination of the statistical probability of a particular biological event or state of interest. Those of skill in the art will appreciate that markers may differ in their specificity and/or sensitivity as related to a particular biological event or state of interest.

The amount of CXCL9 and SPP1 gene transcripts in tumor associated macrophages isolated from a cancer sample can be normalized to the expression level a housekeeping gene (e.g., RPL37A). The amount of CXCL9 and SPP1 gene transcripts in tumor associated macrophages isolated from a cancer sample can be normalized to each other. The normalized levels of CXCL9 in tumor associated macrophages from a cancer sample can be compared with the normalized levels of SPP1 isolated from the same tumor associated macrophages from the same cancer sample to calculate a CXCL9:SPP1 ratio. The amount of CXCL9 gene transcripts and SPP1 gene transcripts in a tumor associated macrophage cancer sample can be determined by any method of gene expression quantification known in the art (e.g., histological/immunohistochemistry, qPCR, RNAseq).

The CXCL9:SPP1 ratio serves as a reliable prognostic biomarker for cancer progression, intervention-responsive cancers (e.g., immunotherapy and chemotherapy), clinical outcome, prognosis, and/or survival of the subject. In some embodiments, the CXCL9:SPP1 ratio reference level is at or greater than 1. A CXCL9:SPP1 ratio in tumor associated macrophages in a cancer sample at or greater than 1 indicates that the cancer can respond to immunotherapy and/or chemotherapy treatments. A CXCL9:SPP1 ratio in tumor associated macrophages in a cancer sample less than 1 indicates that the cancer will not respond to immunotherapy or chemotherapy treatments. Thus, the tumor associated macrophage CXCL9:SPP1 ratio can serve as a biomarker for the methods described herein. Cancer treatment decisions can be made based on a subject's tumor associated macrophage CXCL9:SPP1 ratio, wherein a course of treatment can be determined based on the ratio.

### Methods of Screening

The present methods generate a CXCL9:SPP1 ratio from tumor associated macrophages and then use the CXCL9:SPP1 ratio as a prognostic biomarker to determine clinical outcomes, appropriate cancer therapies, and survivability predictions. The present methods can include screening for intervention-responsive tumors, including measuring the expression level of CXCL9 and SPP1 in tumor associated macrophages from a tumor sample; determining a CXCL9:SPP1 ratio; and identifying the tumor sample as originating from an intervention-responsive tumor if the CXCL9:SPP1 ratio is above a reference level.

A subject can be an individual (e.g., a mammal such as a human) having cancer (e.g., a solid tumor). In some embodiments, the subject may have already undergone cancer treatment (e.g., surgery, radiotherapy, immunotherapy, or chemotherapy), in other embodiments, the subject may be treatment naïve. A sample can be obtained from the subject (e.g., a tumor sample); a sample can be obtained from the subject at multiple time points.

The sample (e.g., tumor sample) can be processed to isolate the tumor associated macrophages present within the sample. The tumor associated macrophages can be cultured prior to analysis of the expression level of genes of the tumor associated macrophages. The tumor associated macrophages can be analyzed to determine the expression level of CXCL9 and SPP1 in the tumor associated macrophages. The expression level can be determined using any methods of quantifying gene expression level known in the art (e.g., northern blot, immunohistochemistry, qPCR, DNA microarray, or RNAseq). The expression level of CXCL9 and SPP1 can be normalized to a housekeeping gene (e.g., any housekeeping gene associated with macrophages), and the normalized levels of CXCL9 and SPP1 can be compared in a ratio of CXCL9 expression to SPP1 expression. The resulting CXCL9:SPP1 ratio can then be used to make judgments about the tumor (e.g., whether the tumor is likely to be responsive to immunotherapy or chemotherapy, whether the tumor is likely to progress), and about the subject (e.g., whether the subject is likely to have a good clinical outcome, whether the subject is likely to survive).

In cases where the methods presented herein indicate that a subject having cancer has an intervention-responsive tumor, the methods can further include administering one or more immunotherapy agents to the subject. Thus, the present methods include administering one or more immunotherapy agents, e.g., immune checkpoint inhibitors, e.g., immune checkpoint inhibitory antibody, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4, or an antibody that binds to T-cell immunoglobulin and ITIM domains (TIGIT). In some embodiments, an anti-PD-1 antibody is used.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. No. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3: 15-25 (2010); Storz, Mabs. 2016 Jan.; 8(1): 10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR 1476.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US PGPub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1): 80-96; Antoni et al., Am Soc Clin Oncol Educ Book. 2016; 35: e450-8; US PGPub. Nos. 20180326054; 20180251767; 20180230431; 20170334995; 20170290914; 20170101472; 20170022273; 20160303124, and include BMS-986016.

Exemplary anti-TIGIT antibodies that can be used in the methods described herein include those that bind to human TIGIT; an exemplary human TIGIT sequence is provided at NCBI Accession No. NP_776160.2. Exemplary antibodies include AB154; MK-7684; BMS-986207; ASP8374; Tiragolumab (MTIG7192A; RG6058); (Etigilimab (OMP-313M32)); 313R12. See, e.g., Harjunpää and Guillerey, Clin Exp Immunol 2019 Dec. 11[Online ahead of print], DOI: 10.1111/cei.13407; 20200062859; and 20200040082.

Further, in cases where the methods presented herein indicate that a subject having cancer has an intervention-responsive tumor, the methods can further include administering one or more chemotherapy agents to the subject. Thus, the present methods include administering one or more chemotherapy agents, e.g., paclitaxel, docetaxel, cisplatin, cyclophosphamide, doxorubicin, 5-fluorouracil, gemcitabine, or trabectedin. See Larionova I, Cherdyntseva N, Liu T, Patysheva M, Rakina M, Kzhyshkowska J. Interaction of tumor-associated macrophages and cancer chemotherapy. Oncoimmunology. 2019 Apr. 13; 8(7): 1596004. doi: 10.1080/2162402X.2019.1596004.

### EXAMPLES

The invention is further described in the following examples, which do not limit the scope of the invention described in the claims. The methods described here were used to generate the examples described herein.

### Human Sample Collection and Processing

Fresh tumor samples for scRNAseq and histological analyses were collected from HNSCC patients with signed informed consent under IRB approved protocols [Massachusetts General Hospital (MGH) (IRB #2014P000559; IRB #13-416) and Massachusetts Eye and Ear (MEE) (IRB #11-024H)]. Fresh HNSCC biopsy specimen HUG-01 (male, primary HPV-negative oropharynx cancer) and HUG-02 (female, HPV-negative larynx cancer) were obtained at the oto-rhino-laryngology department of the Hôpitaux Universitaires de Genève (HUG) after obtaining signed informed consent. The requirement for an ethics approval has been waived by the Geneva state ethics committee (Ref #2022-01356). HPV status was determined based on p16 expression by immunohistochemistry (IHC) and/or by HPV 16/18 in situ hybridization (ISH). In a few cases, HPV diagnostics was complemented with HPV PCR. Nasopharyngeal squamous cell carcinoma cases were tested for EBV by EBER ISH. Clinical demographic information, such as age, sex, smoking, and others, were extracted from electronic medical records. Non-smokers were defined as having a smoking history of <10 pack years. HN23 and HN45 represent subsequent recurrences of the same patient. For patients with scRNAseq analysis of tumor tissue obtained before the start of an anti-PD1 mAb-containing immunotherapy in the palliative setting, response annotation was determined based on clinical-radiological best response. Stable disease was defined as no significant change in tumor burden at around 6 months after initiation of the immunotherapy-containing treatment. Patients with progressive or stable disease were classified as “non-responders”, whereas patients with partial or complete response were classified as “responders”.

### Ex Vivo Culture of HNSCC Single-Cell Dissociates

Fresh HNSCC biopsies (HUG-1 and HUG-2) were immediately transferred into sterile RPMI 1640 (Gibco, Cat #61870-010) supplemented with 1% penicillin/streptomycin (P/S; Gibco, Cat #15140-122) and kept on ice. Single cell suspension was achieved following a protocol by Pollard and colleagues (51). In brief, tissues were cut into small pieces using a scalpel and digested in RPMI1640 with 1% P/S and Liberase DL (0.28 U/ml; Roche, Cat #05401160001), Liberase TL (0.56 U/ml; Roche, Cat #5401020001) and DNase I (0.15 mg/ml; Sigma, Cat #10104159001) and incubated at 37 °C for 45 minutes while shaking at 700 rpm. The digestion was stopped by adding complete culture medium (RPMI 1640, 10% fetal calf serum (FCS; Gibco, Cat #A5256701), 1% P/S) and the suspension was filtered through a 100 μm cell strainer. Cells were centrifuged and the cell pellet was adjusted to 8×105 cells/ml in complete culture medium and 1 ml of the cell suspension (per well) was plated in 24-well plates. Cells were immediately stimulated with 40 ng/ml recombinant human IFN-gamma (Peprotech, Cat #AF-300-02)(25) or subjected to hypoxic conditions (1% O₂) for 3 days. Subsequently, cells were collected using trypsin (0.05%, Gibco, Cat #25300-054), stained for surface antibodies diluted in FACS buffer (PBS, Gibco, Cat #10010-015; 2.5% FBS and 2 mM EDTA, Invitrogen, Cat #15575-038) for 30 min at 4 °C and enriched for TAMs by sorting on a FACS ARIA II (BD Bioscienec) or a MoFlo Astrios EQ (Beckman) living DAPI-CD45+ (CD3/CD19/CD56)-CD66b-cells (CD45 PE, clone HI30, Biolegend, Cat #304058, RRID: AB_2564156; CD3 BV421, clone OKT3, Biolegend, Cat #317344, RRID: AB_2565849; CD19 BV421, clone HIB19, Biolegend, Cat #302234, RRID: AB_11142678; CD56 BV421, clone NCAM16.2, BD Biosciences, Cat #562751, RRID: AB_2732054; CD66b FITC, clone 80H3, Immunotech, Cat #IM0531U) directly into the lysis buffer provided by the RNAeasy Micro kit (Qiagen, Cat #74004) for subsequent RNA isolation and qRT-PCR.

### THP-1 Cell Culture

THP-1 cells (human acute monocytic leukemia cell line, kindly provided by D. Migliorini, University of Geneva) were cultured in suspension in complete Medium (RPMI-1640, 10% FCS, 1% P/S). 2.5×106 THP-1 cells per well were plated into 6-well plates (in technical triplicates) and macrophage-like polarization was induced by stimulation with phorbol-12-myristate-13-acetate (PMA; Sigma-Aldrich, Cat #P8139-5MG) for 72 hours resulting in adhering cells, followed by a resting period of 24 hours after removing the PMA (adapted from (52)). Subsequently, cells were either stimulated with 40 ng/ml recombinant human IFN-gamma (Peprotech, Cat #AF-300-02) or subjected to hypoxic conditions (1% O₂) for 24 hours before cells were harvested for RNA isolation by directly adding Qiazol lysis reagent (Qiagen, Cat #217004) into the wells.

### RNA Isolation and qRT-PCR

Total RNA was isolated using RNAeasy Micro (for FACS sorted TAM-enriched cells; Qiagen, Cat #74004) and miRNAeasy Mini (for THP-1 cells; Qiagen, Cat #217004) kits following the manufacturers recommendations. cDNA was synthesized employing the SuperScript™ VILO™ cDNA Synthesis kit (Thermo Fisher Scientific, Cat #11754050) according to the manufacturers protocol and quantitative PCR (qPCR) was done on a QuantStudio 6 Flex Real-Time PCR system (Thermo Fisher Scientific) and by using the following TaqMan gene expression assays (Thermo Fisher Scientific): Hs00171065_m1 CXCL9, Hs00959010_m1 SPP1, Hs06050959_g1 RPL37A. Expression levels of CXCL9 and SPP1 were normalized to RPL37A. Fold changes were determined employing the comparative Ct method (ΔΔCt). In conditions with undetectable transcript amplification (non-detects), CT values were set to 40.

### CD8 IHC and Analysis

FFPE tissue was cut in 4-5 μm thick sections, which were dried at 55 °C on a heating block overnight, then cooled down to room temperature (RT) for at least 1 hour, and treated with xylene/ethanol for deparaffinization. Hematoxylin&Eosin (H&E) staining was performed using a standard protocol. For IHC, slide pretreatment and staining used reagents provided with the En Vision Flex Mini Kit, High pH (Link; Agilent Dako, Cat #K8023) and following the manufacturer's recommendations. In brief, antigen retrieval (pH 9) was done using a pressure cooker for 2 minutes at 120 °C followed by stepwise cooling. The primary anti-CD8 antibody (clone 4B11, Cat #CD8-4B11-L-CE-H, RRI, Leica Biosystems, RRID: AB 10555292) was incubated overnight at 4 °C at a 1:400 dilution. The incubation times for DAB and the Enhancer were 6 and 5 minutes, respectively. Slides were counterstained with hematoxylin. The CD8 antibody was previously validated and titrated on normal tonsil sections and antibody specificity and staining quality were assured by board certified pathologists (P.M.S. and W.C.F.). The entire tissue cross-sections were imaged and digitalized with a digital slide scanner NanoZoomer 2.0RS (Hamamatsu) at 40× and images were analyzed by NDP.view2 software (Hamamatsu Photonics KK). The tumor area was identified on an H&E-stained adjacent tissue section by a board-certified pathologist (P.M.S.). Whenever possible, 6 representative ROIs per sample with an area of 0.2 mm2 at the interface of tumor nests and surrounding stroma were defined. CD8+ cells were manually counted and results were displayed as number of CD8+ cells per mm2.

### Multiplexed Immunofluorescence

Four μm thick FFPE sections of 28 tumors matching the tumors analyzed by scRNAseq were stained by multiplexed immunofluorescence (IF). Twenty-three samples passed quality control and could be used for subsequent analysis. Multiplexed staining was performed on automated Ventana Discovery Ultra staining module (Ventana, Roche, Serial #313236) using a preset protocol. Slides were baked at 60 °C for 1 hour, placed on the staining module for deparaffinization (Discovery Wash, Roche, Cat #950-510), and in high pH for 64 min at 95 °C for epitope retrieval (Discovery CC1, Roche, Cat #950-500). Multiplex staining consisted in multiple rounds of staining. Each round included endogenous peroxidase quenching (Discovery Inhibitor, Roche, Cat #760-4840), non-specific sites blocking for 8 minutes (Discovery Goat IgG and Discovery Inhibitor, Roche, Cat #760-6008), primary antibody incubation, secondary HRP-labeled antibody incubation for 16 minutes (OmniMap anti-rabbit HRP, Roche, Cat #760-4311, RRID: AB_2811043, or OmniMap anti-mouse HRP, Roche, Cat #760-4310, RRID: AB_2885182), OPAL™ reactive fluorophore detection (Akoya Biosciences, Marlborough, MS, USA) that covalently label the primary epitope, and heat denaturation of antibodies (Discovery CC2, Roche, Cat #950-223). The sequence of antibodies used in the multiplex assay with the associated Opal dyes is as follows: 1) Rabbit anti-CD4 1 h, 37 °C (Cat #104R-16, Cellmarque, RRID: AB_1516770)/Opal690; 2) Rabbit monoclonal anti-FoxP3 1 h at 37 °C (Cat #ab99963, abcam, RRID: AB_10675258)/Opal620; 3) Rabbit monoclonal anti-PD-1, 1 hour at RT (Cat #ab137132, abcam, RRID: AB_2894867)/Opal570; 4) Rabbit monoclonal anti-TCF1, 1 h at RT (Cat #2203s, Cell Signaling, RRID: AB_2199302)/Opal520; 5) Mouse monoclonal anti-cytokeratin, 1 hour at 37 °C (Cat #M3515, Dako, RRID: AB_2132885)/Opal480; 6) Rabbit monoclonal anti-CD8, 1 hour at 37 °C (Cat #108R-16, Cellmarque, RRID: AB_2892088). Nuclei were visualized by a final incubation with Spectral DAPI (1/10, Akoya Biosciences, Cat #FP1490) for 12 minutes. The slides were mounted with fluorescence mounting medium (Cat #S3023, Dako), stored in the dark at 4 °C, and scanned within 48 hours.

### Combined RNA In Situ Hybridization-Immunofluorescence

For combined RNA-ISH-IF (RNAscope) analysis, samples with high or low CS TAM polarity (defined by scRNAseq analysis) were selected. CShi TAM samples: HN25, HN28, HN30, HN52, HN74. CSlo TAM samples: HN13, HN38, HN45, HN57, HN66. For individual experiments, either all or some of these were analyzed. Four um thick sections from FFPE blocks were used and an in-situ hybridization (ISH) with co-immunostaining (IF) was performed following the manufacturer's recommendation of the RNAscope Multiplex Fluorescent Reagent Kit v2 assay (Cat #323100, Advanced Cell Diagnostics). Combined ISH and antibody staining with Opal dyes (Akoya Biosciences) was performed manually for ISH and employing an automated Ventana Discovery Ultra Staining module (Ventana, Roche) for IF. For individual experiments, the following combinations of RNAscope probes (all from Biotechne) and antibodies were employed. All Opal dyes were purchased from Perkin Elmer AG:
i) Probe-Hs-CXCL9 probe (Cat #440161) with Opal570 (Cat #FP1488001KT), Probe-Hs-SPP1-C2 probe (Cat #420101-C2) with Opal690 (Cat #FP1497001KT), anti-CD68 Ab (Cat #M0876, Dako, RRID: AB_2074844) with Opal520 (Cat #FP1487001KT) and anti-pan-cytokeratin Ab (Cat #ab9377, Abcam, RRID: AB_307222) with Opal620 (Cat #FP1497001KT);
ii) Probe-Hs-CXCL9 probe (Cat #440161) with Opal690 (Cat #FP1497001KT), Probe-Hs-IFNG-C2 probe (Cat #310501-C2) with Opal570 (Cat #FP1488001KT), anti-CD3 Ab (Cat #A0452, Dako, RRID: AB_2335677) with Opal620 (Cat #FP1497001KT) and anti-CD68 Ab (Cat #M0876, Dako, RRID: AB 2074844) with Opal520 (Cat #FP1487001KT).
iii) Probe-Hs-IFNG probe (Cat #310501) with Opal570 (Cat #FP1488001KT), Probe-Hs-SPP1-C2 probe (Cat #420101-C2) with Opal690 (Cat #FP1497001KT), anti-CD68 Ab (Cat #M0876, Dako, RRID: AB_2074844) with Opal520 (Cat #FP1487001KT) and anti-GLUT-1 antibody (HPA031345, Sigma, RRID: AB 2673835) with Opal620 (Cat #FP1497001KT).
iv) Probe-Hs-CXCL9 probe (Cat #440161) with Opal690 (Cat #FP1497001KT), Probe-Hs-CXCR3-C2 (Cat #539251-C2) with Opal570 (Cat #FP1488001KT), anti-CD68 Ab (Cat #M0876, Dako, RRID: AB_2074844) with Opal520 (Cat #FP1487001KT) and anti-CD3 Ab (Cat #A0452, Dako, RRID: AB_2335677) with Opal620 (Cat #FP1497001KT).
v) Probe-Hs-ITGB1 probe (Cat #516931) with Opal570 (Cat #FP1488001KT), Probe-Hs-SPP1-C2 probe (Cat #420101-C2) with Opal690 (Cat #FP1497001KT), anti-CD68 Ab (Cat #M0876, Dako, RRID: AB_2074844) with Opal520 (Cat #FP1487001KT) and anti-Fibroblast activation protein (FAP) Ab (Cat #ab207178, Abcam, RRID: AB_2864720) with Opal620 (Cat #FP1497001KT).

Nuclei were visualized by final incubation with Spectral DAPI (1/10, Cat #FP1490, Akoya Biosciences). The slides were mounted with fluorescence mounting medium (Cat #S3023, Dako) and stored in the dark at 4 °C until scanned within 48 h. Images were acquired on the Vectra Polaris automated imaging system at 10X× and 40× (Akoya Biosciences), and qualitatively analyzed using Phenochart (Akoya Biosciences).

### Multispectral Imaging and Data Analysis

Multiplex immunofluorescence (IF) images were acquired at 20× and 40× on the Vectra® Polaris automated quantitative pathology imaging system (Akoya Biosciences, Marlborough, USA), allowing the unmixing of spectrally overlapping fluorophores and tissue autofluorescence of whole slide scans. For optimal signal unmixing (individual spectral peaks) and subsequent multiplex analysis, a spectral library containing the individual emitting spectral peaks of all 7 fluorophores was created and validated using the inForm Analysis software (Akoya Biosciences). Phenotypic analysis was performed using inForm 2.5.1 image analysis software (Akoya Biosciences) enabling a per-cell idenfication of weak and/or spectrally overlapping IF markers of multiplex-stained tissue sections. The images were first segmented into tissue areas of tumor, stroma and no tissue, based on panCK and DAPI staining patterns, using the inForm Tissue Finder™ algorithms. Individual cells were segmented using the counterstained-based adaptive cell segmentation algorithm. Quantification of immune cells was performed using the inForm active learning phenotyping algorithm by assigning different cell phenotypes across several images representative of the whole scan. InForm software was trained to recognize cell phenotypes according to combination of probes/Ab described above. This algorithm was applied on the whole scan by batch to quantify all the different cell types and a custom R-script was used to retrieve all combined cell phenotypes and scoring into tabulated data files.

### Spatial Analysis of Histological Data

Plotting of “pseudo-image” (2D scatter plot of cell-level biomarker status in histological coordinates) and neighborhood statistical analysis were done under R statistical software using custom package (gitlab.com/pwirapati/spati). Briefly, the neighborhoods and cell layers were defined based on adjacency matrix derived from Delaunay triangulation after filtering edges longer than 50 μm (which typically correspond to non-biological adjacency around borders or empty interiors). The neighborhood size around a cell is defined by the number of layers (one level of breadth-first search on the adjacency graph). Four layers were used for all analyses, which balance the local resolution with sufficient number of cells to give reliable cell counts. A typical four-layer neighborhood contains approximately 70 cells, with the average distance from the outer layer to the center of around 40 to 50 μm. Statistical tests for enrichment is done by counting the percentage of target cells (as the dependent variable) in the neighborhoods surrounding the cell type to be compared (the independent variable). The logarithm of the ratio of the target cell to total cell in a neighborhood (with a shift of 0.5 to moderate for low counts) is used as the response variable in linear models comparing the different groups of the central cell of interest, with standard statistical tests of significance (which gives very similar p-values to permutation tests of central cell labels). The exponential of the coefficient in a two-group comparison is used as fold-change summary. For the analysis of cell distribution with respect to tumor-stoma interface, first the general tumor versus stroma areas were identified by morphological closing followed by morphological opening (53) applied to tumor marker status as binary state of the cells (which can be dichotomized cytokeratin status in RNA-ISH-IF or assigned tumor labels based on clustering in CosMx). The cell layers inwards and outwards following the contour of the tumor-stoma interface were identified by successive morphological erosions (inside the tumor area) and dilations (outside in the stromal area). The number of erosions and dilations to reach a layer was interpreted as the distance to the interface (approximately 10 μm between layers). Cell frequency statistics were calculated for cells with the same layer number from the entire tissue sample.

### Public Spatial Transcriptomics Data

The dataset from (47) was used as provided by the authors, including the cell type classifications. Only the data from lung cancer patient 13 was used, due to sufficiently high number and balanced presence of CXCL9+ and SPP1+ cells required for the relevant comparisons. For dichotomizing the gene expression into “+” or “−” cells, the gene-specific modes of the distribution of non-zero values were used for the cutoffs.

### Processing of Fresh Tissue for scRNAseq

All samples in this study were rapidly dissociated after the surgical procedure using the Human Tumor Dissociation kit from Miltenyi Biotec (Cat #130-095-929), with minor modifications to the protocol. First, the tissue was placed into 1.5 ml tubes containing 420 μl DMEM and minced into small fragments (1-2 mm) using surgical scissors, followed by the addition of 42 μl of enzyme H, 21 μl of enzyme R, 5 μl of enzyme A (all provided in the kit), and 512 μl of DMEM (reaching a total volume of 1 ml). Second, the minced tissue was vortexed for 2 seconds and then immediately placed into a thermomixer (Eppendorf, F1.5) for 15 minutes at 37 °C, 350 rpm. Next, tissues were placed on a 50 μm filter (Cat #04-004-2327, Sysmex), minced with a 1 ml syringe plunger, and washed with 10 ml of DMEM containing 10% heat-inactivated FCS. After spinning the tube at 1500 rpm at 4 °C for 5 minutes, the supernatant was removed, and ACK buffer (Cat #A1049201, Gibco) was added to ensure the removal of red blood cells (RBCs). Following RBC lysis, the dissociated sample was resuspended in DMEM with 10% FCS and the number and viability of the cells was determined using a manual hemocytometer (Cat #1492, Bright-line). If the sample's viability was below 70%, the Easy Sep Dead Cell Removal (Annexin V) kit (Cat #17899, STEMCELL) was used, with minor modifications to the protocol, allowing cell viability enrichment with a low input of cells. Briefly, cells were washed with 1 ml of RT isolation media (PBSx1+2% FCS+1 mM CaCl₂), resuspended in 100 μl, and moved into a PCR 8-strip well. Next, 10 μl dead cell removal (Annexin V) and 10 μl of biotin selection cocktails (provided with the kit) were added, gently mixed with the cell suspension, and incubated for 4 minutes at RT. After the first incubation, 20 μl of vortexed RapidSpheres and 60 μl of isolation media were added, mixed, and incubated for 3 minutes at RT. Lastly, the PCR 8-strip tube containing the cells was placed on a 10× Genomics magnetic separator (Cat #120250). After a 5-minute incubation at RT, the supernatant containing the viable cells was moved into a new 1.5 ml collection tube, span for 5 minutes at 1500 rpm, 4 °C; resuspended in DMEM with 10% FCS (for 10× Genomics GEM generation and barcoding) and counted using a manual hemocytometer.

### Single-Cell Library Generation and Sequencing

Generation of single-cell whole transcriptome libraries was done using the 10× Genomics Chromium Single Cell 5 'Library and Gel Bead Kit v1 chemistry (Cat #1000006, 10× Genomics;) with Chromium Single Cell 5 'Feature Barcode Library kit (Cat #1000080, 10× Genomics) according to the manufacturer instructions. Quality control (QC) was performed for each sample after generating cDNA, using the Qubit dsDNA High Sensitivity kit (Cat #Q32854, Invitrogen) and the Agilent High Sensitivity BioA DNA kit (Cat #5067-4626, Agilent), for concentration and size distribution evaluation respectively. Only samples that have passed QC were used for library generation and were sequenced on a NextSeq 500 sequencer (Illumina) using pair-end reads, with 26 reads for read 1 and 55 reads for read 2.

### Single-Cell RNA Pre-Processing and Cell Annotation

scRNA alignment and quantification were done using CellRanger (version 3.1.0). QC and cell filtering were done using R package Seurat (54), based on multiple summaries (total read counts and expressed genes, mitochondrial fraction) as well as custom rules based on marker genes to identify doublets and to rescue neutrophils. The following criteria were used to filter out unwanted cells: (1) number of detected genes <500 (low quality) or >5000 (doublets), (2) >20% mitochondrial genes, (3) cells expressing tumor and immune markers (doublets). From the discarded subset, neutrophils were rescued by identifying those expressing any one of the neutrophil markers: FCGR3B, CXCR1, CXCR2, PI3, G052, or CSF3R. InferCNV was used to distinguish tumor versus non-tumor epithelial cells. The cell annotation pipeline combined the FindCluster method in Seurat's package with visualization of UMAP coordinates using Spring viewer (55) to facilitate manual examination and cell type assignment. The process was iterative done at multiple levels: total cells to detect major compartment and lineages, followed by subclustering for more refined subdivisions into “minor states”. T cells subclustering was also using projecTILs (33). Some minor state clusters were discarded when clear biological meaning could not be attributed (for example, clustering driven by cell cycle genes). Biologically related minor states were combined into larger group as required by the context of the analysis.

### Patient-Based Single-Cell Aggregation

The expression of a specific gene in a cell type and a patient was summarized by averaging the raw counts (the sum of read counts divided by the number of cells). Summary profiles from different patients were normalized in cell-type specific manner. The profile for a patient in a cell type was scaled by dividing by the mean (across all genes) and multiplying by the mean of mean profiles (across all patients). This quantity is referred to as “adjusted mean counts”. The calculation and visualization as “dot plots” (such as FIG. 2E) were carried out using a custom R package (https://gitlab.com/pwirapati/acdx).

### Dominant Marker Genes Analysis

To identify genes with enriched expression in a particular cell type, the per-patient summary expression data were averaged (in log scale) for any given gene and cell type, and differential gene expression was calculated as the lowest between all pairs of comparisons and between a given cell type and all other cell types. Genes were selected based on calculating significance test statistics and multiple testing FWER (p <0.05) using the bootstrap resampling method (56), and required a fold-change of at least 3 when compared to all other cell types. A total of 1189 genes fulfilled these criteria.

Public bulk mRNA data, prognostic signature and survival analysis TCGA HNSCC datasets (57) were obtained from UCSC Xena (58). Dataset FHUT GSE41613 (59) and LPZG GSE65858 (60) were downloaded from NCBI Gene Expression Omnibus. Only common genes were used, and gene-mean centering per dataset were done to correct for batch effects and platform differences before combining the cohorts. HNSCC subtypes were determined using the signatures published in Walter et al (21). Missing HPV status were predicted using a signature constructed from the data where they are available, using a binomial model in glmnet (61).

To identify genes that, within a given cell type, were related to survival, we performed cell-type-specific prognostic signature analyses. These signatures were constructed by selecting prognostic gene-pair log ratios using a procedure similar to that of Paquet and Hallett (22) but with Cox regression significance as the score. The relevant pairs were selected and combined using the Cox model in GLMNET (23), using an elastic net parameter of 0.1 to allow comprehensive gene inclusion. Double cross-validation was used for selecting the penalty factor (inner loop) and performance assessment (outer loop). Results were based on outer-cross validation. Among the prognostic gene pairs identified in macrophages, we selected CXCL9 and SPP1because: (i) this pair alone was sufficient to capture the entire TAM signature information; (ii) CXCL9 and SPP1 already have established and arguably opposing roles in cancer biology (25, 26, 24); (iii) access to expression of these genes was widely available on all platforms used for bioinformatics analysis of external datasets (in addition, these genes can generate a strong signal in these analyses); (iv) CXCL9 and SPP1 have the advantage of both being listed in current databases as having multiple partners, which was considered useful for ligand-receptor interaction analyses; and (v) for spatial analysis using RNAscope, probes for these two genes were already available and provided good labeling performance (it is not necessarily easy to obtain such high-quality probes for arbitrary genes). CXCL9 and SPP1 were convenient choices for this study, but other genes associated with CS TAM polarity could also have roles in shaping the TME and merit further investigation.

For bulk mRNA survival analysis, pan-cancer TCGA datasets were obtained from UCSC Xena. CXCL9/SPP1 ratios (or difference in log scale) were based on provided normalized and combined data. FIG. 2E shows that (1) macrophages are the main expressers of CXCL9 and SPP1, (2) cell types other than macrophages can also express these genes albeit at lower levels, (3) the expression level of these two genes is co-regulated between the different cell populations. Indeed, CXCL9 and SPP1 expression in TAMs is strongly correlated with total CXCL9 and SPP1 expression in bulk RNAseq data (R=0.87 for CXCL9, R=0.81 for SPP1). Thus, the CS ratio in bulk data can be interpreted as an indicator of the CS ratio in macrophages.

### Between-Cell Type Co-Regulation Analysis

Spearman's rank correlation was performed between CS TAM and the per-patient expression summary in other cell types for all genes that passed filtering criteria (average expression >0.1 reads/cell). False discovery rate 0.05 and correlation >0.55 were used for significant cell type-gene combinations in MGH/MEE-HNSCC. Geneset enrichment was done using R package fgsea (62) separately for each cell type, using the Spearman's correlation against CS TAM as the ranking score.

### Ligand-Receptor Pair Analysis

We used the curated ligand: receptor database included in the NicheNet package (35), excluding predicted subsets (ppi_prediction and ppi_prediction_go) and including well-established KEGG, guide2pharmacology and Ramilowski subsets. Based on these lists, a ligand: receptor pair was considered of interest if: 1) both the ligand and the receptor were expressed, and 2) expression of the ligand and/or the receptor correlated with CS TAM polarity. Circos plots were made using R package circlize (63) with custom scripts.

### scRNAseq Analysis of Publicly Available Validation Cohorts

scRNAseq datasets of the independent HNSCC (UPit-HNSCC, GSE164690, (44)), NSCLC (GSE131907, Kim et al., 2020, (45)), and CRC (GSE178341, (46)) cohorts were obtained from NCBI GEO. The cell annotations from the original authors were used when available (NSCLC and CRC). Manual calling was done on UPit-HNSCC, based on clustering according to dominant genes identified in MGH-HNSCC (FIG. 1B). Patient-based summarization was done in the same way as for the MGH-HNSCC cohort, based on the published scRNAseq raw counts.

### Statistical Analysis

Various statistical tests, regression fitting and plotting were done in R statistical software (www.R-project.org). All correlations are Spearman's rank correlation. Linear fits were performed using 1 m( ), applied on shifted log-transformation where appropriate (such for abundance and gene expression data). Kaplan-Meier plots, univariate and multivariate Cox regression were done using coxph function in R package survival. All statistical tests were two-sided (unless otherwise indicated, such as in dominant marker gene analysis). Statistical significance was at the level of p<0.05, with appropriate multiple testing applied depending on the context. Other details of statistical methods for specific analyses are described in each sections above.

### Example 1: Tumor-Associated Macrophages are Major Contributors to the Clinical Outcome of HNSCCs

A scRNAseq dataset was generated from 52 fresh HNSCC tumor tissues from 51 individual patients (MGH/MEE-HNSCC cohort) with a wide range of clinical characteristics, spanning the clinical spectrum, from primary tumors to locoregional recurrences to distant metastases, and without selection or enrichment for a particular cell type before sequencing. Cell clusters were defined from a total of 187,399 cell transcriptomes, using Seurat's FindCluster and manually annotating them at different resolutions, from main cell compartments to minor cell states. This dataset comprehensively covered all three main cell compartments, namely tumor, stromal and immune cells, as well as all major cell types, namely tumor and epithelial cells, fibroblasts, endothelial cells, lymphocytes and myeloid cells (FIG. 1A). These cell types were reproducibly found in patients, but their abundance varied greatly.

Epithelial cells, fibroblasts, endothelial cells, lymphocytes, and myeloid cell states contained a finite number of minor cell states, which were also reproducibly found in patients; this was in stark contrast to the tumor cell states from the same patients, which were largely patient-specific.

1189 genes were identified whose expression in the MGH/MEE-HNSCC cohort was dominant (i.e., at least 3-fold higher) in a given cellular compartment relative to all others (FIG. 1B). This gene list was used to interpret a collection of bulk mRNAseq data from 886 HNSCC patients obtained by pooling three independent cohorts (TCGA, LPZG, FHUT). The expression heat-map of these genes in all 886 patients (FIG. 1B) showed mixing of the cohorts, indicating inter-cohort homogeneity. It also showed an association between human papilloma virus (HPV) status and molecular tumor subtype, as expected (21). Finally, pairwise correlation analysis showed that the TME compartment-dominant genes were co-enriched in their respective cellular compartments (FIG. 1B, right), indicating that what becomes apparent as co-regulation in bulk mRNA data may in fact largely be driven by heterogeneity in abundance of various TME cell types. Remarkably, tumor cell-gene intrinsic clustering was still associated with differential abundance of stromal and immune cell transcriptomes, suggesting the existence of an orchestrated cellular communication between different TME compartments.

Using the information obtained from the analyses above, the influence of the three main cellular compartments (tumor, stroma, immune) on clinical outcome was investigated. Supervised methods that are commonly used for bulk mRNAseq analysis were applied, but considered only the cell-type dominant genes as the feature variables. To ensure internal normalization of gene expression in each cell type, a pairwise log ratio method (22) was used followed by construction of multi-pair signatures using an elastic network classifier (23) that was trained on overall patient survival. Univariate analysis showed that each major cellular compartment had a significant prognostic impact. However, a multivariate analysis in which all three compartments compete to explain the patient's clinical course showed that only the stromal (p=0.01) and immune (p=0.0003) signatures retained their prognostic ability (FIG. 1C), indicating a more direct association between these non-tumor components with disease progression.

Because immune cells include different subsets with distinct functions, which cells carried the most prognostic information was determined. In univariate analyses, every major immune cell type except monocytes had a significant prognostic impact; however, in multivariate analyses only mast cell (p=0.04) and macrophage (p=0.01) signatures retained independent effects (FIG. 1D). This showed that prognostic information from the different cell types was mostly redundant and suggested close coordination between these cells. We also illustrated the relevance of the macrophage-specific signature in Kaplan-Meier survival analyses (FIG. 1E).

Among the prognostic gene pairs identified in macrophages, we selected CXCL9 and SPP1 because the expression ratio of these two genes (CS) sufficiently captured the information in the entire signature (FIG. 1K) and these two genes already have established and arguably opposing roles in cancer biology (24-26). The association of the other gene pairs with survival is shown in FIG. 1K. CS tumor-associated macrophage (TAM) polarity was associated with overall survival when analyzed separately in the three independent HNSCC cohorts (FIG. 1L-M); it also remained linked to disease outcome independently of CD274 expression (FIG. 1N), which is frequently considered predictive of a patient's clinical course (27). Furthermore, CS TAM polarity remained independent of tumor HPV status and disease stage. Taken together, these data indicated that the TAM gene expression programs were particularly important in defining disease progression.

### Example 2: CXCL9 and SPP1 Identify Tumor-Associated Macrophage Polarity In Vivo

Quantitative analyses of scRNAseq (FIG. 1F) and histological (FIG. 1G) data both showed that expression of CXCL9 and SPP1 by macrophages was largely mutually exclusive at the single cell level. Also, by inferring the abundance of TAMs and their CS polarity in bulk RNAseq data, we found that CS polarity was prognostic, whereas TAM abundance was not (FIG. 1H). Patients with more SPP1^{hi} TAMs had poorer clinical outcomes, whereas patients with more CXCL9^{hi} TAMs had better ones and the CS ratio correlated strongly with our initial macrophage prognostic score; in contrast, TAM expression of M1 (ITGAX and CD80) or M2 (CD163 and MRC1) macrophage markers, which are widely used to define macrophages with anti- and pro-tumor functions, respectively, was not related to distinct clinical outcome in the same patients (FIG. 1I). These results support the notion that M1 and M2 phenotypes define the possible extremes of polarized cells in vitro but may not necessarily reflect the complexity of TAMs in vivo (28). Finally, CS TAM polarity was low pretreatment in nine out of nine patients who did not respond to anti-PD1-containing treatment regimens, but high in three out of five patients who responded to treatment (FIG. 1J). This is additional evidence that CS TAM polarity alone may carry meaningful clinical information.

Next, whether CXCL9 and/or SPP1 expression was restricted to discrete, minor monocyte or TAM states was evaluated. To this end, unsupervised clustering of scRNAseq data was used to reveal the complexity of monocytes and macrophages in HNSCCs. Stereotyped gene expression programs that defined a total of four conserved monocyte states and seven conserved TAM states was found, which were reproducibly found in patients. Each state was named after the gene that it expressed dominantly over all other monocyte and TAM states. Monocytes mostly expressed low levels of CXCL9 and SPP1. Among TAMs, Mac_APOE, Mac_CXCL10, and Mac_F13A1 preferentially expressed CXCL9, and Mac_SPP1 and Mac MTIH preferentially expressed SPP1; however, all states of TAMs expressed both transcripts at variable levels (these findings are further discussed below and presented in FIG. 2F).

Whether any of the observed monocyte or TAM states corresponded to M1 or M2 cells was also examined. To this end, M0, M1 and M2 signatures of in vitro-polarized cells were used, involving a total of 596 genes identified from public dataset GSE158094 (29). SPP1^{hi} and CXCL9^{hi} TAMs all expressed elevated M0, M1 and M2signatures compared to monocytes and Mac_MARK4. The only substantial differences were higher M2 vs M1 signatures in Mac_SPP1 and higher M1 vs M2 signatures in Mac_CXCL10. These results align with previous findings suggesting that the phenotypes of human TAMs only partially overlap with those of M1 and M2 markers (18, 28, 30, 31). Therefore, the study of CXCL9 and SPP1 expression by TAMs should be more relevant to capture the polarity of these cells in vivo and their association with other TME components and disease progression.

### Example 3: CXCL9/SPP1 Tumor-Associated Macrophage Polarity Defines the Broader HNSCC Microenvironment

For subsequent analyses, the CS ratio was used to assess TAM polarity in vivo (FIG. 2A). Furthermore, tumor-to-tumor variation of TAM polarity was examined, considering that this variation may reveal rules governing the cellular and molecular composition of the TME. Specifically, we classified the 52 samples according to CS TAM polarity and performed correlation analyses between this variable and other TME variables, including the abundance of different cell types and their gene expression profiles. For the latter, the main unit of analysis was the expression of a gene averaged over all cells from the same sample and cell state. The resulting value is independent of cell abundance, unlike “pseudo-bulk” sums that are typically used (32). In doing so, co-regulation of gene expression between cell types was detected, which indicates the existence of a coordinated communication network in the TME, as detailed below.

CS TAM polarity was positively associated with increased tumor infiltration by three major immune cell types, namely T-cells, B-cells and DCs (FIG. 2B). Operationally dividing T-cells into well-defined transcriptional states (33) revealed that all of them contributed to the association between CS TAM polarity and overall T-cell abundance. Histological analyses on tissue sections (n=23) derived from the same tumors that were analyzed by scRNAseq confirmed this finding for various CD4⁺ and CD8⁺ T-cell populations (FIG. 2C). Similarly, both plasma and non-plasma B-cells contributed to the association between CS TAM polarity and overall B-cell abundance. On the other hand, operationally dividing intratumoral DCs into well-defined states (34) showed that only the abundance of type 1 conventional DCs (cDC1_XCR1) was associated with CS TAM polarity. Also, CS TAM polarity did not associate with the abundance of other stromal or immune cell types, including TAMs themselves (FIG. 2B). Therefore, the quality of TAMs, not their quantity, was related to the intratumoral abundance of three cell types, namely T-cells, B-cells and DCs, all of which have been linked to antitumor immunity (2).

The scRNAseq data further showed that M1 or M2 markers, in contrast to CXCL9 and SPP1, were not strongly associated with T-cell, B-cell or DC abundance in the same tumors (FIG. 2D). Histological staining of CD8⁺ T-cells in tumors confirmed these findings. Overall, these data confirmed the existence of a coordination between CS TAM polarity and adaptive immune responses in human HNSCC; they also indicated the limited link between traditional M1 and M2 markers and such immune responses.

The analyses of tumor-to-tumor variation was extended in CS TAM polarity by investigating its connection to gene expression in all TME cell types. Initially, CXCL9 was assessed and found that not only TAMs but also tumor cells, fibroblasts, endothelial cells, monocytes and DCs could express it, despite at lower levels (FIG. 2E). Importantly, all these cell types did so in a coordinated manner with TAMs. For example, patients with higher CXCL9 expression in TAMs also had higher CXCL9 expression in these other cell types. In a like manner, tumor cells and monocytes could express SPP1 at lower levels; they did so in a coordinated manner with TAMs, and opposite to CXCL9 expression (FIG. 2E). This harmonized gene expression across cell types was independent of key tumor and patient characteristics, namely HPV/EBV status, nature of specimen obtained, anatomic site of tumor origin, sex, and smoking. Together, these results suggested that CS TAM polarity could broadly inform on related activities of other cells of the TME.

All conserved minor macrophage states expressed both CXCL9 and SPP1; they did so at varying levels but in a coordinated manner (FIG. 2F). For example, Mac_CXCL10 cells expressed CXCL9 at the highest level on average, but all other minor macrophage states expressed CXCL9 to some degree. The level of CXCL9 expression in Mac_CXCL10 cells varied between patients, but the inter-patient variation was preserved in all other minor macrophage states. Analysis of other minor cell states indicated that the results obtained for macrophages were broadly generalizable. For example, among monocytes, Mo_LYPD2 and Mo CCL3L1expressed CXCL9 in a manner that was coordinated with each other and with that of macrophages; similarly, Mo_CD300LB and Mo_CCL3L1 expressed SPP1 in a coordinated manner. The same findings applied to DCs: cDC1_XCR1, DC3_LAMP3 and cDC2/MoDC_CLEC10A all expressed CXCL9 in a manner that was coordinated with that of macrophages. Minor states that did not follow this coordination rule were generally those in which the gene of interest was never expressed; for example, Mo_PADI4, Mo_CD300LB, cDC2_CD1A and pDC_LILRA4 largely lacked CXCL9 expression in the 52 tumor samples analyzed. CXCL9 and SPP1 were primarily expressed in macrophages, with expression of both genes coordinated across macrophage states and, more broadly, across various immune and nonimmune cells in the TME.

Next, whether CS TAM polarity was co-dependent on that of other transcripts was investigated. To this end, we again studied all TME cell types. We found that 2,320 cell type-gene combinations (including 1,812 unique genes) were significantly associated with CS TAM polarity (FDR<0.05). The expression of a few of these genes, such as STAT1, was globally shared among all immune and nonimmune cell types (FIG. 2E). The coordinated expression of STAT1 in different cell types suggests the existence of coordinated programs that can occur across the entire TME and correlate with CS TAM polarity; yet cell type-specific programs correlating with CS TAM may be directly or indirectly linked to STAT1 activity, and possibly influenced by STAT1-independent pathways. The expression of other genes was instead coordinated with CS TAM polarity only in certain cell types, such as CXCR6 in T-cells and SLC2A1 (GLUT1) in tumor cells and macrophages (FIG. 2E). In addition, CS TAM polarity was directly associated with the expression level of a large set of cytokines and cytokine receptors, either positively (i.e., genes expressed more strongly in CS^{hi} tumors, such as CXCL9, CXCL10, CXCL11, CXCL16, and IL7) or negatively (i.e., genes expressed more strongly in CS^{lo} tumors, such as CXCL5, CXCL8, IL1A, IL1B and IL1RN) (FIG. 2G). The abundance of other genes, such as IFNG, CCL5, and CXCL13, was indirectly correlated with CS TAM polarity, as it depended on the abundance of the cell state expressing this gene. Finally, some genes were coordinated with CS TAM polarity only in certain minor cell types; such as IL12B in DC LAMP3 but no other DC state. Overall, this coordination of expression of many genes, all connected to CS TAM polarity, occurred simultaneously in virtually all TME cell types.

To determine whether the genes associated with CS TAM polarity globally contributed to distinct biological processes, gene set enrichment analyses in each cell type separately was performed. This revealed not only which gene expression programs were associated with CS TAM polarity, but also in which cell types these programs were active (FIG. 2H). Cs^{hi} TAM-associated pathways included immune-related IFNα and IFNγ signaling in almost all cell types; IL-6/JAK/STAT3 signaling in tumor cells, fibroblasts, and endothelial cells; and complement activity in tumor cells, fibroblasts, and macrophages. In contrast, CS^{lo} TAM-associated pathways included epithelial-mesenchymal transition, hypoxia, and mTOR signaling in almost all cell types; glycolysis and angiogenesis in tumor cells, myeloid cells and T-cells; TNFα signaling in myeloid cells and T-cells; and TGFβ signaling in monocytes, mast cells and T-cells. Taken together, these data linked CS TAM polarity to a variety of immune and nonimmune cell programs, all of which have been independently proposed to regulate tumor growth in a positive or negative manner, and which we reveal here to be deeply interconnected in human tumors.

To identify possible cellular interactions involved in this broad program of co-regulation associated with TAMs, we used the well-curated subset of ligand: receptor pairs obtained from the NicheNet database (35), and only considered those for which the expression of the ligand and/or the receptor correlated with that of CXCL9 or SPP1 in TAMs. In addition, we manually annotated these predicted interactions a priori as putatively immuno-activating, immuno-inhibiting, or unknown, based on the nature of the role of the ligand or receptor reported in the literature. This analysis identified multiple ligand: receptor interactions associated with CXCL9^{hi} TAMs, many of which were putatively immuno-activating and did not involve TAMs (FIG. 2I, left). In particular, we found predicted interactions between DCs and T-cells through the ligand: receptor pairs IL12B:IL12RB1, CXCL16:CXCR6, XCL1:XCR1, and CXCL9:CXCR3. All of these molecular interactions involving tumor-associated DCs have been shown to promote local antitumor T-cell functions in experimental mouse models (25, 36-39). We also found predicted interactions between TAMs and T-cells through the CXCL9:CXCR3 pair. The ability of macrophage-derived CXCL9 to stimulate antitumor T immunity has been shown in mice (40). CXCL9 expression in TAMs was also associated with putatively immuno-inhibiting CD274:PDCD1 (PD-L1:PD1) interactions between tumor cells and T-cells. This is consistent with our findings that tumors with CS^{hi} TAMs were enriched in IFNγ signaling and that CD274 is an IFNγ inducible gene (41). Overall, the CXCL9-associated interaction network was strongly associated with inflammatory reactions and antitumor immunity. The interaction network associated with SPP1^{hi} TAMs was more limited than that associated with CXCL9^{hi} TAMs and focused less on immune cell interactions (FIG. 2I, right). SPP was expressed in TAMs and tumor cells, whereas known SPP1 receptors (integrins and CD44) were found in a variety of cell types, such as fibroblasts and endothelial cells, consistent with the role of SPP1 in metastasis and angiogenesis (42). Since SPP1 is an extracellular matrix protein, activation of the SPP1-associated interaction network in tumors may reflect a shift from tissue inflammation to tissue repair.

To refine the above results, interactions at the level of minor cell states were investigated. Interactions of some ligand:receptor pairs, such as CXCL9:CXCR3, CXCL16:CXCR6, and CD40LG:CD40, were rather pleiotropic because they involved many different minor cell states (with interesting new details, such as some minor DC states potentially acting as both sender and receiver in CXCL9:CXCR3 interactions). In contrast, other ligand:receptor pairs, such as XCL1:XCR1 and IL12B:IL12RB1, connected only certain minor cellular states. For instance, XCL1:XCR1 interactions selectively involved cDC1s as a sensor of the chemokine XCL1, which is known to recruit cDC1s to the TME and promote immune control of cancer (38). Similarly, IL12B:IL12RB1 interactions selectively involved DC_LAMP3 DCs (previously identified as DC3 (18, 34), mregDC (20), and LAMP3⁺ DC (17)) as producers of IL-12, which can locally stimulate antitumor immunity (36).

These data indicate that CS TAM polarity identifies critical cellular and molecular TME programs; these programs have been independently recognized for their ability to regulate tumor growth, and we show here they are all connected and identifiable by CS TAM polarity.

### Example 4: Regulation of CXCL9/SPP1 Tumor-Associated Macrophage Polarity

One possible explanation for the largely mutually exclusive expression of CXCL9 and SPP1 in TAMs could be that the tissue niche provides discrete cell-extrinsic cues that promote the generation of CXCL9⁺ or SPP1⁺ TAMs. Thus, whether these cells distributed differently in tumors was investigated. Histological analyses, combining RNA in situ hybridization (RNA-ISH) with immunofluorescence (IF), not only confirmed the CS TAM polarity of tumors initially identified by scRNAseq (FIG. 3L), but also showed that TAMs accumulated often as clusters of either CXCL9⁺ or SPP1⁺ cells (FIGS. 3A-B). The distribution of CXCL9⁺ and SPP1⁺ TAMs in the tumor stroma and tumor nests varied among patients, although there were increased accumulations of CXCL9⁺ or SPP1⁺ TAMs at the interface between the tumor stroma and tumor nests in CS^{hi} or CS^{lo} patients, respectively. In contrast, CXCL9⁻ SPP1⁻ TAMs were more found in the tumor stroma and further away from the interface between stroma and nest. Additional histological analyses showed that tumor cells expressing CXCL9 or SPP1 were more likely to be found at the vicinity of CXCL9⁺ or SPP1⁺ TAMs, respectively (FIGS. 3C-D). These data align with the identification of a coordinated regulation of CXCL9 and SPP1 expression between different cell types in the same tumors (FIG. 2E); they also suggest that local cues may foster the acquisition of CXCL9⁺ or SPP1⁺ phenotypes.

To examine whether distinct environmental signals might foster the production of CXCL9⁺ or SPP1⁺ TAMs, cellular and molecular neighbors of these two cell populations were characterized. CD3⁺ and CXCR3⁺ cells were enriched near CXCL9⁺ TAMs, which validated the ligand: receptor analysis results suggesting CXCR3:CXCL9 interactions between T-cells and macrophages (FIG. 2I), and aligned with previous findings in mice linking CXCR3:CXCL9 signaling to intratumoral antitumor immunity (39). Furthermore, the neighborhood of CXCL9⁺ TAMs was enriched of IFNG⁺ T-cells (FIGS. 3E-F), which represented the vast majority of IFNG-producing cells (FIG. 3G). Conversely, the neighborhood of SPP1⁺ TAMs was enriched in GLUT1⁺ cells (FIGS. 3H-I) indicating hypoxic areas (43). Taken together, the data indicate that CXCL9⁺ and SPP1⁺ TAMs exist in distinct neighborhoods, which contain factors that are linked to tumor control (T-cells, IFNγ) and tumor progression (hypoxia), respectively, in broad agreement with the results of non-spatial scRNAseq analysis (FIGS. 2E-G).

Given the physical proximity between CXCL9⁺ TAMs and CXCR3⁺ IFNG⁺ T-cells in vivo, and the ability of IFNγ to stimulate CXCL9 expression in other cancer settings (25), in vitro testing of whether exposing macrophages to IFNγ alone would promote the acquisition of a CXCL9⁺ phenotype was conducted. Conversely, given the physical proximity between SPP1⁺ TAMs and hypoxic areas identified by GLUT1 positivity, and the association between CS^{lo} TAM polarity and hypoxia signatures, we tested whether hypoxia would promote the acquisition of an SPP1⁺ phenotype in macrophages in vitro. To test these hypotheses, THP-1 cells were differentiated into macrophage-like cells (FIG. 3J). Exposure to IFNγ promoted CXCL9 expression, whereas hypoxic conditions promoted SPP1 expression; consequently, IFNγ increased the CS macrophage ratio, whereas hypoxic conditions decreased it. To extend these analyses to TAMs, we generated single cell dissociations of primary human HNSCCs biopsies (FIG. 3K). Again, exposure to IFNγ promoted CXCL9 expression and increased the CS macrophage ratio, whereas hypoxic conditions promoted SPP1 expression and decreased the CS macrophage ratio. These results indicate that certain microenvironmental factors, namely IFNγ and hypoxia, can respectively regulate CXCL9 and SPP1 expression in TAMs. These results also help explain the spatial segregation of CXCL9⁺ and SPP1⁺ TAMs in tumors.

### Example 5: Relevance of CXCL9/SPP1 Tumor-Associated Macrophage Polarity in a Variety of Cancers

Using a pan-cancer approach exploiting bulk RNAseq data from >10,000 patients (TCGA pooled data), we found that CS TAM polarity was strongly associated with overall survival in this combined cohort (FIG. 4J). This association was particularly notable for patients with liver, ovarian, cervical, thyroid, or bladder cancer, as well as melanoma and sarcoma, in addition to head and neck cancer as shown above (FIG. 4K). These data confirmed that CS TAM polarity alone carries significant clinical information; they also suggested that the relevance of this polarity to understanding the inner workings of the TME extends to many cancer indications.

To widen analyses to scRNAseq data, CS TAM polarity in an independent HNSCC cohort (n=18 patients (44)) was interrogated, as well as in non-small cell lung cancer (NSCLC; n=37 patients (45)) and colorectal cancer (CRC; n=62 patients (46)) cohorts. These cohorts were chosen because they included a sufficiently large number of patients to examine tumor-to-tumor variations. For each cohort, patients were ranked according to CS TAM polarity, and then analyzed various cell types in the TME. In all three newly analyzed datasets, CXCL9 expression in TAMs was inversely correlated with SPP1 expression in the same cells (FIG. 4A), recapitulating our initial results obtained with the MGH/MEE-HNSCC cohort (FIG. 1F). Therefore, the expression of CXCL9 and SPP1 may be used to define TAM polarity in different cancer types. In marked contrast, monocytes and macrophages found in adjacent normal tissue were all largely CXCL9 and SPP1 (FIG. 4B), further suggesting that the acquisition of a CXCL9⁺ or SPP1⁺ phenotype requires environmental signals that may be absent in normal tissues.

Analyzing all cell types revealed that in addition to TAMs, tumor cells and fibroblasts could also express CXCL9, although at lower levels. Moreover, these cells did so in a manner coordinated with TAMs. Tumor cells and monocytes could also express SPP1 and did so in a way that was consistent with TAMs, and inversely with CXCL9 expression. Fibroblasts, and to a lesser extent DCs, mast cells, T-cells, and B-cells, expressed SPP1 in a manner coordinated with TAMs in the different cohorts analyzed, suggesting a possible extension of the synchronized expression of CXCL9 and SPP1 in the TME to these cells. Whether CS TAM polarity was co-dependent with that of other transcripts in the same patient cohorts was investigated and presented above. Each cell type was analyzed separately to reveal genes in each that might be coordinately expressed with CS polarity in TAMs; this approach identified the existence of CS TAM co-regulatory programs in virtually all cell types and in all cohorts. By performing a “correlation of correlations” analysis, we confirmed that the strong association between CS TAM and cell type-specific gene expression initially identified in the MGH/MEE-HNSCC cohort was largely replicated in the other three cohorts (FIG. 4C). In addition, CS TAM polarity correlated with the abundance of T-cells, B-cells and DCs in these cohorts. The correlations were particularly significant for T-cells and DCs in the CRC cohort, which contained a larger number of patients, and for all three cell types in all cohorts combined (FIG. 4D). Thus, in all cancer indications that we were able to analyze, CS TAM polarity was linked to (i) the abundance of antitumor cell types, and (ii) critical gene expression profiles in virtually all immune and nonimmune cells of the TME.

To examine the spatial distribution of CXCL9⁺ or SPP1⁺ TAMs in another cancer indication, single cell spatial transcriptomics of tissue sections were obtained from lung adenocarcinoma patients (47). TAMs could accumulate as clusters of either CXCL9⁺ or SPP1⁺ cells (FIG. 4E). These data were confirmed with two other approaches using the CellCharter algorithm (48): first, unsupervised identification of spatial clusters centered on TAMs indicated two major hubs, which were enriched in CXCL9⁺ and SPP1⁺ TAMs, respectively; second, neighborhood enrichment analysis revealed that SPP1⁺ TAMs were depleted near CXCL9⁺ TAMs and vice versa. Furthermore, SPP1⁺ TAMs were more surrounded by SLC2A1⁺ (GLUT1) cells, whereas CXCL9⁺ TAMs had more CXCR3⁺ and IFNG⁺ T-cells near them (FIG. 4E). These data indicate that, irrespective of the cancer indication, CXCL9⁺ and SPP1⁺ TAMs may exist in distinct neighborhoods, which contain factors that are linked to tumor control (T-cells, IFNγ) and tumor progression (hypoxia), respectively.

To further assess the relevance of CS polarity, it was examined whether its association with clinical outcome was dependent, or not, of cancer-associated variables. These analyses used bulk RNAseq data from 33 pooled cancer entities (n=10,429 patients), from whom the clinical outcome was known. Univariate analyses identified that both cancer stage and the CS ratio were associated with clinical outcome. Furthermore, bivariate analysis showed that both variables remained significantly associated with clinical outcome. These data indicate that CS polarity was independent of cancer stage across cancer types, thereby extending our initial findings in HNSCC. Using a similar approach, CS polarity was found to be independent of known EMT markers linked to poor prognosis (49), both in HNSCC (FIG. 4F) and in combined cancer indications (FIG. 4G). Similarly, we CS polarity, interferon and effector T-cell signatures (49) were compared and associated with clinical outcome. IFNG expression on its own, as well as of CD8A expression as a proxy for a CTL response was considered. Univariate analyses identified that each variable was associated with a better clinical outcome in HNSCC, with the exception of IFNG alone whose hazard ratio was just not significant. However, bivariate analyses revealed that the interferon and T-cell signatures were all competed by CS polarity (FIG. 4H), suggesting that the latter is superior and sufficient to identify at least some TIL processes, e.g., those involving IFNγ responses. These findings were extended to all combined cancer indications (FIG. 4I), further indicating that CS TAM polarity alone carries meaningful clinical information in multiple cancer types.

### REFERENCES

1. M. Binnewies et al., Nat Med 24, 541 (2018).
2. D. Bruni, H. K. Angell, J. Galon, Nat Rev Cancer 20, 662 (2020).
3. J. Galon et al., Science 313, 1960 (2006).
4. S. M. Mahmoud et al., J Clin Oncol 29, 1949 (2011).
5. N. Nguyen et al., Head Neck 38, 1074 (2016).
6. F. Petitprez et al., Nature 577, 556 (2020).
7. R. Cabrita et al., Nature 577, 561 (2020).
8. A. Italiano et al., Nat Med 28, 1199 (2022).
9. M. L. Broz et al., Cancer Cell 26, 638 (2014).
10. J. Goc et al., Cancer Res 74, 705 (2014).
11. M. C. Dieu-Nosjean et al., J Clin Oncol 26, 4410 (2008).
12. Q. W. Zhang et al., PLoS One 7, e50946 (2012).
13. C. Steidl et al., N Engl J Med 362, 875 (2010).
14. R. Noy, J. W. Pollard, Immunity 41, 49 (2014).
15. A. P. Patel et al., Science 344, 1396 (2014).
16. S. V. Puram et al., Cell 171, 1611 (2017).
17. Q. Zhang et al., Cell 179, 829 (2019).
18. R. Zilionis et al., Immunity 50, 1317 (2019).
19. H. Li et al., Cell 176, 775 (2019).
20. B. Maier et al., Nature 580, 257 (2020).
21. V. Walter et al., PLoS One 8, e56823 (2013).
22. E. R. Paquet, M. T. Hallett, J Natl Cancer Inst 107, 357 (2015).
23. N. Simon, J. Friedman, T. Hastie, R. Tibshirani, J Stat Softw 39, 1 (2011).
24. R. Tokunaga et al., Cancer Treat Rev 63, 40 (2018).
25. D. Dangaj et al., Cancer Cell 35, 885 (2019).
26. S. S. McAllister et al., Cell 133, 994 (2008).
27. B. Burtness et al., Lancet 394, 1915 (2019).
28. M. J. Pittet, O. Michielin, D. Migliorini, Nat Rev Clin Oncol 19, 402 (2022).
29. S. X. Liu, H. H. Gustafson, D. L. Jackson, S. H. Pun, C. Trapnell, Sci Rep 10, 12273 (2020).
30. M. Orecchioni, Y. Ghosheh, A. B. Pramod, K. Ley, Front Immunol 10, 1084 (2019).
31. S. Cheng et al., Cell 184, 792 (2021).
32. H. L. Crowell et al., Nat Commun 11, 6077 (2020).
33. M. Andreatta et al., Nat Commun 12, 2965 (2021).
34. G. M. Gerhard, R. Bill, M. Messemaker, A. M. Klein, M. J. Pittet, J Exp Med 218, (2021).
35. R. Browaeys, W. Saelens, Y. Saeys, Nat Methods 17, 159 (2020).
36. C. S. Garris et al., Immunity 49, 1148 (2018).
37. M. Di Pilato et al., Cell 184, 4512 (2021).
38. J. P. Böttcher et al., Cell 172, 1022 (2018).
39. M. T. Chow et al., Immunity 50, 1498 (2019).
40. I. G. House et al., Clin Cancer Res 26, 487 (2020).
41. C. Sun, R. Mezzadra, T. N. Schumacher, Immunity 48, 434 (2018).
42. C. Hao et al., Int J Mol Med 39, 1327 (2017).
43. M. Pliszka, L. Szablewski, Cancers (Basel) 13, 4184 (2021).
44. C. H. L. Kürten et al., Nat Commun 12, 7338 (2021).
45. N. Kim et al., Nat Commun 11, 2285 (2020).
46. K. Pelka et al., Cell 184, 4734 (2021).
47. S. He et al., Nat Biotechnol 40, 1794 (2022).
48. M. Varrone, D. Tavernari, A. Santamaria-Martínez, G. Ciriello, bioRxiv 2023.01.10.523386 (2023).
49. A. Bagaev et al., Cancer Cell 39, 845 (2021).
50. T. A. Wynn, A. Chawla, J. W. Pollard, Nature 496, 445 (2013).
51. L. Cassetta et al., Adv Exp Med Biol 899, 211 (2016).
52. W. Chanput, J. J. Mes, H. J. Wichers, Int Immunopharmacol 23, 37 (2014).
53. J. Serra, Image Analysis and Mathematical Morphology (Academic Press, London, 1982).
54. R. Satija, J. A. Farrell, D. Gennert, A. F. Schier, A. Regev, Nat Biotechnol 33, 495 (2015).
55. C. Weinreb, S. Wolock, A. M. Klein, Bioinformatics 34, 1246 (2018).
56. P. H. Westfall, S. S. Young, Resampling-based Multiple Testing: Examples and Methods for p-value Adjustment (Wiley, New York, 1993).
57. C. G. A. Network, Nature 517, 576 (2015).
58. M. J. Goldman et al., Nat Biotechnol 38, 675 (2020).
59 P. Lohavanichbutr et al., Clin Cancer Res 19, 1197 (2013).
60. G. Wichmann et al., Int J Cancer 137, 2846 (2015).
61. J. Friedman, T. Hastie, R. Tibshirani, J Stat Softw 33, 1 (2010).
62. G. Korotkevich et al., bioRxiv (2021).
63. Z. Gu, L. Gu, R. Eils, M. Schlesner, B. Brors, Bioinformatics 30, 2811 (2014).

1. A method of screening for intervention-responsive tumors, the method comprising:
(a) measuring the expression level of CXCL9 and SPP1 in tumor associated macrophages from a tumor sample;
(b) determining a CXCL9:SPP1 ratio;
(c) identifying the tumor sample as originating from an intervention-responsive tumor if the CXCL9:SPP1 ratio is above a reference level; and
(d) administering to the subject an immunotherapy agent.

2. A method of determining a ratio between CXCL9 and SPP1 in a tumor sample, the method comprising:
(a) obtaining a tumor sample;
(b) isolating the tumor associated macrophages from the tumor sample;
(c) determining expression levels of CXCL9 and SPP1 in the tumor associated macrophages;
(d) normalizing expression levels of CXCL9 and SPP1 compared to a housekeeping gene; and
(e) comparing the normalized expression level of CXCL9 to the normalized expression level of SPP1 to generate a CXCL9:SPP1 ratio.

3. A method of predicting cancer progression in a subject, the method comprising:
(a) measuring the expression level of CXCL9 and SPP1 in tumor associated macrophages from a tumor sample;
(b) determining a CXCL9:SPP1 ratio;
(c) identifying the tumor sample as originating from an intervention-responsive tumor if the CXCL9:SPP1 ratio is above a reference level; and
(d) using the intervention-responsive tumor status to predict cancer progression in a subject with cancer.

4. (canceled)

5. The method of claim 1, wherein the method consists of measuring the expression level of CXCL9 and SPP1 in tumor associated macrophages.

6. The method of claim 1, further comprising obtaining a tumor sample from the subject.

7. The method of claim 6, wherein the sample comprises a solid tumor.

8. The method of claim 1, wherein the cancer is a solid tumor cancer.

9. The method of claim 8, wherein the solid tumor cancer comprises a sarcoma, a carcinoma, a lymphoma, a germ cell tumor, or a blastoma.

10. The method of claim 1, wherein the cancer comprises a chondrosarcoma, a leiomyosarcoma, a rhabdomyosarcoma, a mesothelial sarcoma or mesothelioma, a fibrosarcoma, a angiosarcoma or hemangioendothelioma, a liposarcoma, a glioma, an astrocytoma, a myxosarcoma, a mesenchymouse or mixed mesodermal cancer.

11. The method of claim 1, wherein the cancer comprises an adenocarcinoma or a squamous cell carcinoma.

12. The method of claim 1, wherein the cancer is a skin cancer, a brain cancer, a liver cancer, a pancreatic cancer, a kidney cancer, a bladder cancer, a thyroid cancer, a stomach cancer, a lung cancer, a head or neck cancer, an eye cancer, a lip or oral cancer, a gastrointestinal cancer, a colorectal cancer, an anal cancer, a breast cancer, a cervical cancer, an endometrial cancer, an ovarian cancer, an esophageal cancer, a thymoma, a bone cancer, a lymph node cancer, a prostate cancer, a testicular cancer, a penile cancer, a gallbladder cancer, a uterine sarcoma, an adrenal gland cancer, an ampullary cancer, a nasal or a paranasal sinus cancer.

13. The method of claim 1, wherein the cancer is a head or neck cancer.

14. The method of claim 1, wherein the CXCL9:SPP1 ratio reference level is at or greater than 1.

15. The method of claim 1, wherein the expression levels of CXCL9 and SPP1 are normalized to a housekeeping gene prior to determining the CXCL9:SPP1 ratio.

16. The method of claim 15, wherein determining the CXCL9:SPP1 ratio comprises comparing the normalized level of CXCL9 expression to the normalized level of SPP1 expression in the sample.

17. The method of claim 1, wherein the expression level of CXCL9 and SPP1 is determined by immunohistochemical methods.

18. The method of claim 1, wherein the expression level of CXCL9 and SPP1 is determined by qPCR.

19. (canceled)

20. The method of claim 1 wherein, the immunotherapy agent is a checkpoint blockade immunotherapy agent.

21. The method of claim 20, wherein the checkpoint blockade immunotherapy agent is an anti-PD1 immunotherapy agent.
